(19) United States
(12) Patent Application Publication
(10) Pub. No.: US 2011/0044937 A1
(43) Pub. Date: Feb. 24, 2011

(54) STAGED IMMUNE-RESPONSE MODULATION IN ONCOLYTIC THERAPY

(75) Inventors: John C. Bell, Ottawa (CA); Caroline Judith Breitbach, Kirkland (CA); Harry Atkins, Orleans (CA)

Correspondence Address: FULBRIGHT & JAWORSKI L.L.P., 600 CONGRESS AVE., SUITE 2400, AUSTIN, TX, 78701, US

(73) Assignee: OTTAWA HEALTH RESEARCH INSTITUTE, San Francisco, CA (US)

(21) Appl. No.: 12/375,361

(22) PCT Filed: Jul. 27, 2007
(86) PCT No.: PCT/CA07/01340

§ 371 Date: Oct. 14, 2010

Related U.S. Application Data
(60) Provisional application No. 60/820,601, filed on Jul. 27, 2006.
(60) Provisional application No. 60/898,998, filed on Feb. 2, 2007.

(51) Int. Cl. A61K 35/76; A61K 39/395; A61K 38/20; A61K 38/19; A61K 38/21; A61P 35/00

(52) U.S. Cl. 424/85.2; 424/93.6; 424/173.1; 424/85.1; 424/85.5

(57) ABSTRACT

The invention provides methods for treating tumours, such as solid tumours, in a host. The methods may involve infecting the tumour with an amount of one or more strains of oncolytic virus. The virus will generally be selected to be effective to cause a lytic infection of tumour cells within the tumour. In various embodiments, the host neutrophil response to the lytic infection may be modulated, so that during the course of the lytic infection, the host has an initial neutrophil response and a secondary neutrophil response, these two responses being different in some material respect. For example, the secondary neutrophil response may mediate a greater degree of apoptotic killing of tumour cells than does the initial neutrophil response.

### FIELD

The invention is in the field of cancer treatment, particularly oncolytic viral therapies.

### BACKGROUND

A wide variety of oncolytic viruses have been used in preclinical and clinical cancer therapies (see Parato at al., 2005; Bell at al, 2003; Everts and van der Poel, 2005; Ries and Brandts, 2004). For example, an improved therapeutic response has been reported in patients suffering from squamous cell cancer who receive a combination of oncolytic virus therapy and chemotherapy, compared to patients who receive chemotherapy alone (Xia et al., 2004). Oncolytic viruses that have been selected or engineered to productively infect tumour cells include adenovirus (Xia et al., 2004; Wakimoto et al., 2004); reovirus; herpes simplex virus 1 (Shah, et al., 2003); Newcastle disease virus (NDV; Pecora, et al., 2002); vaccinia virus (Mastrangelo et al.,1999; US 2006/0099224); coxsackievirus; measles virus; vesicular stomatitis virus (Stojdl, et al., 2000; Stojdl, et al., 2003); influenza virus; myxoma virus (Myers, R. et al., 2005). For example, EP 1218019, US 2004/208849, US 2004/115170, WO 2001/019380, WO 2002/050304, WO 2002/043647 and US 2004/170607 disclose oncolytic viruses, such as Rhabdovirus, picornavirus, and vesicular stomatitis virus (VSV), in which the virus may exhibit differential susceptibility, particularly for tumor cells having low PKR activity. WO 2005/007824 discloses oncolytic vaccinia viruses and their use for selective destruction of cancer cells, which may exhibit a reduced ability to inhibit the antiviral dsRNA dependent protein kinase (PKR) and increased sensitivity to interferon. WO 2003/008586 similarly discloses methods for engineering oncolytic viruses, which involve alteration or deletion of a viral anti-PKR activity. WO 2002/091997, US 2005/208024 and US 2003/77819 disclose oncolytic virus therapies in which a combination of leukocytes and an oncolytic virus in suspension may be administered to a patient. WO 2005/087931 discloses selected Picornavirus adapted for lytically infecting a cell in the absence of intercellular adhesion molecule-1 (ICAM-1). WO 2005/002607 discloses the use of oncolytic viruses to treat neoplasms having activated PP2A-like or Ras activities, including combinations of more than one type and/or strain of oncolytic viruses, such as reovirus. US 2006/18836 discloses methods for treating p53-negative human tumor cells with the Herefordshire strain of Newcastle disease virus. WO 2005/049845, WO 2001/053506, US 2004/120928, WO 2003/082200, EP 1252323 and US 2004/9604 disclose herpes viruses such as HSV, which may have improved oncolytic and/or gene delivery capabilities.

In many instances, oncolytic viral vectors have been administered by intratumoural injection, such as vectors based on vaccinia virus, adenovirus, reovirus, newcastle disease virus, coxsackievirus and herpes simplex virus (HSV) (Shah et al., 2003; Kaufman, et al. 2005; Chiocca et al., 2004; Harrow at al., 2004; Mastrangelo et al., 1999). In metastatic disease, a systemic route of delivery for oncolytic viruses may be desirable, for example by intravenous administration (Reid et al., 2002; Lorence et al., 2003; Pecora et al., 2002; Lorence et al., 2005; Reid at al., 2001; McCart et al., 2001).

Although systemic administration of oncolytic viruses may be desireable, this exposes the virus to heightened immune surveillance. It has accordingly been suggested that oncolytic viral therapy might be facilitated by ablation or attenuation of the patient's immune system, which for example occurs during radiation therapy and chemotherapy for cancer (Parato et al., 2005). In mouse tumour model studies with reovirus, HSV and adenovirus oncolytic vectors, it has been shown that antitumour efficacy can be increased by treatment with the chemotherapeutic agent cyclophosphamide, which inhibits neutralizing antibody production (Ikeda et al., 2002; Hirasawa, et al., 2003; Ikeda, K. et al., 1999; Ilan, et al., 1997; Jooss et al., 1996; Kuriyama, et al., 1999; Smith et al., 1996; Wakimoto et al., 2004). US 2006/39894 discloses oncolytic herpes simplex virus strains engineered to counter an innate host immune response. The virus is engineered for expression of the Us11 gene product during the immediate-early phase of the viral life-cycle, preferably without inactivating the Us12 gene, to preserve the ability of the virus to inhibit the host-acquired immune response. Similarly ‘cloaking’ strategies have been proposed to allow a virus to evade the adaptive immune response, such as a vaccinia virus having an extracellular envelope (Ichihashi, 1996) or an adenovirus having a coating of polyethylene glycine or other polymers, or encapsulated with liposomes (Law & Smith, 2001; Fisher, et al., 2001; Holterman et al., 2004; Fukuhara et al., 2003; Eto et al., 2005; Croyle et a, 2001).

There is, however, some degree of risk inherent in using an immunosuppressive regime in conjunction with the therapeutic use of a live virus for oncolytic cancer therapy. In addition, an important component of the long-term therapeutic benefit of at least some oncolytic virus therapeutics may involve activation of the host anti-tumour immune response. For instance, HSV oncolytic therapy is reported to be more effective in immune competent mouse tumour models than in nude mice (Toda et al., 1999; Endo et al., 2002; Toda et al., 2002). Systemic treatment with HSV reportedly leads to both humoral and cellular long term anti-tumour immunity against a breast cancer cell (Hummel et al., 2005). Increases in long-term anti-tumour immunity have been documented following therapeutic treatment with HSV and VSV (Toda et al., 1999; Endo et al., 2002) and a similar phenomenon has been reported with certain vaccinia strains (Parato et al., 2005). it has accordingly been suggested that there may be advantages associated with up-regulating an immune response in conjunction with oncolytic therapy. For example, US 2003/44386 discloses recombinant VSV, expressing cytokines, for the treatment of tumors. Similarly, WO 96/34625 discloses recombinant VSV vectors encoding an interferon, capable of stimulating an immune response. U.S. Pat. No. 6,093,700 discloses methods of inducing an immune response using vaccinia virus recombinants encoding GM-CSF. U.S. Pat. No. 6,475,999 (US 2003/086906) discloses methods of inducing an immune response using vaccinia virus recombinants capable of inducing expression of a selected cytokine.

Neutrophil activation/stimulation is thought to be necessary for the development of effective neutrophil-driven immune responses, for example to pathogens such as bacteria. In natural infections, neutrophils are thought to be one of the first cell types recruited. Accordingly, there are a wide variety of compositions and methods available for stimulating neutrophil activation. For example, US 2005/96259 and WO 2005/041891 disclose a method for activating neutrophils through the use of a neutrophil-activating immune response modifier (IRM) compound and/or a toll-like receptor (TLR)-8-selective agonist. U.S. Pat. No. 6,383,479 and WO1989/004836 disclose the amino acid sequence for biologically-active neutrophil-activating factor (NAF), itself a naturally occurring activating factor for neutrophil cells. WO 1989/004325 discloses a neutrophil-activating polypeptide isolated from human mononuclear cells (i.e., a neutrophil source) that has a molecular weight of 10 kDa. WO 1990/006321 discloses a novel protein factor structurally and functionally related to NAF that has neutrophil-stimulating activity. EP 538030 discloses a novel protein factor, termed ENA-78, that has neutrophil-activating ability. U.S. Pat. No. 5,401,651 and U.S. Pat. No. 5,591,718 disclose methods of identifying inhibitors of ENA-78 which could be used to attenuate neutrophil activation. U.S. Pat. No. 5,759,533 discloses peptide motifs that have neutrophil-stimulating activity and are structurally related to NAF. Similarly, WO 2001/066734 discloses polypeptides isolated from swine heart that have neutrophil-stimulating activity. US 2004/147599 and WO 2002/083120 disclose a composition and method whereby medium-chain fatty acids, glycerides, and analogues promote neutrophil activation. WO 2004/084928 discloses a method and composition comprising peptides S100A8, S100A9, S100A12 or S100A8/A9, for activating neutrophils in immuno-suppressed individuals afflicted with neutropenia (i.e., those individuals with low levels of neutrophils). Grote et al. (2003) disclosed that an attenuated viral infection in which granulocyte-macrophage colony stimulating factor (GM-CSF) is expressed can result in increased neutrophil activation. Jablonska et al. (2002) disclosed that GM-CSF, interferon-γ, and tumor necrosis factor (TNF)-α can activate neutrophils. Likewise, McClenahan et al. (2000) disclosed that TNF-α and PAF (platelet activating factor) can activate bovine neutrophils.

Although activation of neutrophils may be advantageous in some settings, neutrophils are also thought to be involved in various facets of pathological inflammation, such as lung tissue injury following dsRNA administration as well as injury following ischemia/reperfusion (Eltzschig and Collard, 2004; Jiang et al., 2005; Kokura et al., 2002; Vinten-Johansen, 2004). Accordingly, there are a wide variety of compositions and methods available for suppressing neutrophil activity. For example, EP 731709, U.S. Pat. No. 5,709,141, U.S. Pat. No. 5,747,296, U.S. Pat. No. 5,789,178, U.S. Pat. No. 5,919,900, U.S. Pat. No. 6,756,211, U.S. Pat. No. 6,818,616, U.S. Pat. No. 6,962,795, WO 1993/023063, and WO 1994/014973 disclose that compositions enriched for neutrophil inhibitory factor (NIF) inhibit adhesion to vascular endothelial cells and, as such, can be used as a therapy for abnormal inflammatory responses; as disclosed therein, such compositions may contain a glycoprotein isolated from a nematode, particularly that of the genus, Ancylostoma. EP 1238669, US 2002/128230, and U.S. Pat. No. 6,627,621 disclose that the use of glucosamine salts are effective for the inhibition of neutrophil functions and, as such, can be used to treat diseases typified by excessive neutrophilic release of active oxygen and antibiotic proteins. US 2002/159971 and WO 2002/066057 disclose that neutralization of a neutrophil-secreted matrix metalloproteinase (MMP), more specifically MMP-9, can be used to modulate both acute and chronic neutrophil-mediated inflammation. U.S. Pat. No. 5,079,228 discloses that peptides derived from the amino acid sequence of neutrophil activating factor (NAF) can affect neutrophilic chemotaxis through antagonistic effects on native NAF. WO 1992/005796 discloses that administration of an antibody capable of binding to the CD11b subunit of the neutrophil integrin Mo1 (CD11b/CD18) can be used to treat neutrophil-mediated inflammatory damage. U.S. Pat. No. 5,300,292 discloses methods for reducing migration of neutrophils into a tissue comprising administering an effective, inflammation-inhibiting amount of a composition comprising IL-6, or IL-6 and TGFβ. U.S. Pat. No. 5,994,402 discloses methods to reduce or inhibit neutrophil sequestration at an inflammation site using agents such as diethylmaleate (DEM), phorone, buthionine-sulfoximine (BSO), glutathione depleting diethylmaleate (DEM) mimetics, glutathione depleting phorone mimetics and glutathione depleting buthionine sulfoximine (BSO) mimetics. U.S. Pat. No. 6,462,020 discloses peptides having the formula f-Met-Leu-X, where X is selected from the group consisting of Tyr, Tyr-Phe, Phe-Phe and Phe-Tyr, for reducing adhesion, migration or aggregation of neutrophils at a site of inflammation. WO 1995/029243 discloses recombinant proteins having the I-Domain from the human leukocyte beta2-integrin Mac-1, useful for interfering with the cell adhesion mechanism to block adhesion and migration of neutrophils. Tazawa et al. (2003) have disclosed that neutrophils can be depleted through intraperitoneal (i.p.) administration of a monoclonal anti-granulocyte antibody; as disclosed therein, such i.p. treatment can attenuate inflammation-associated carcinogenesis. Onai et al. (2003) have disclosed that intravenous treatment with a small molecule selectin antagonist can attenuate neutrophil-associated myocardial inflammation. Londhe et al. (2005) have disclosed that inhibition of a chemokine ligand/receptor complex, namely CXCR2L/CXCR2, can downregulate neutrophil chemotaxis, a necessary requirement for neutrophil-mediated inflammation. Yasui at al. (2005) have disclosed that treatment with thalidomide can result in the downregulation of neutrophil-based immune responses based on the suppression of NF-κB activation. Grigoryants at al. (2005) have disclosed that treatment with tamoxifen can result in an inhibition of vessel wall neutrophilic immune infiltration. Benjamim et al. (2005) have disclosed that treatment with a cysteinyl-leukotriene receptor antagonist can result in decreased neutrophil inflammation in an experimental model of sepsis. Souza et al. (2003) have disclosed that treatment with a platelet activating factor-receptor antagonist can result in decreased neutrophilic immune cell inflammation.

In keeping with the voluminous art relating to neutrophil stimulation, activation or inhibition (see Morgan et al., 2004), various methods are available for determining the degree of neutrophil activity in a host, as for example is disclosed in U.S. Pat. No. 5,529,907.

It is well established that the solid tumour microenvironment may in some cases become hypoxic (Williams et al. (2005); Okunieff at al. (2005); Cairns at al. (2006)). Accordingly, a wide variety of compounds are available for the specific treatment of hypoxic tumours. For example, dihydropyrimido-quinoxalines and dihydropyrimido-pyridopyrazines (WO 1993/000904); quinoxaline or pyridopyrazine derivatives (WO 1994/006797; WO 1994/006798); 1,2-dihydro-8-piperazinyl-4-phenylimidazopyridopyrazine oxides and 1,2-dihydro-8-piperazinyl-4-phenylimidazo quinoxaline oxides (WO 1993/000900); nitrophenyl mustard and nitrophenylaziridine alcohols, and their corresponding phosphates (WO 2005/042471); Anthraquinone compounds (WO 2005/061453); ligands based on alkylene amine oxime particularly butylene amine oxime ring structures, and radiometal complexes thereof (WO 1995/004552); 1,2,4 benzotriazine 1,4 dioxide compounds (WO 2005/082867); nitro-substituted aromatic or hetero-aromatic compounds (EP 319329).

### SUMMARY

In one aspect, the invention relates to the demonstration that a host neutrophil response may attenuate an oncolytic infection of a tumour. Another aspect of the invention relates to the countervailing demonstration that a host neutrophil response may augment the killing of tumour cells in the course of an oncolytic infection. Combining these effects, the invention, in various aspects, relates to methods of modulating a neutrophil response, including modulating the effects of a neutrophil response, to minimize the attenuation of an oncolytic infection, while optimizing the killing of tumour cells in the course of the infection. The host neutrophil response may accordingly be modulated so that, at the outset of oncolytic treatment, the extent to which the neutrophil response attenuates viral infectivity is reduced. In the course of the oncolytic infection, the neutrophil response may then be modulated to facilitate or augment neutrophil mediated apoptotic killing of tumour cells.

In various aspects, the invention provides methods for treating tumours, such as solid tumours, in a host. The methods may involve infecting the tumour with an amount of one or more strains of oncolytic virus. The virus will generally be selected to be effective to cause a lytic infection of tumour cells within the tumour. In various embodiments, the host neutrophil response to the lytic infection may be modulated, so that during the course of the lytic infection, the host has an initial neutrophil response and a secondary neutrophil response, these two responses being different in some material respect. For example, the secondary neutrophil response may mediate a greater degree of apoptotic killing of tumour cells than does the initial neutrophil response.

In alternative embodiments, the invention may involve suppressing a host neutrophil response to an oncolytic infection. The suppression may for example be effected so that the host has a suppressed neutrophil condition or activity during the initial neutrophil response to the oncolytic infection. For example, an aspect or effect of the host neutrophil response, such as clotting in the tumour vasculature, may be suppressed. As demonstrated herein, this suppression may be modulated so as to increase the number of tumour cells infected with the oncolytic virus during the initial neutrophil response, compared to the number that would be infected in the absence of the step of suppressing the host neutrophil response.

In alternative embodiments, the invention may involve releasing a suppression of the host neutrophil response during the course of the lytic infection. This release of neutrophil suppression may thereby initiate a secondary neutrophil response, so as to facilitate neutrophil mediated inflammation in the tumour during the secondary neutrophil response. As demonstrated herein, this neutrophil mediated inflammation may be modulated so that it results in the apoptotic killing of tumour cells.

In alternative embodiments, the invention may involve stimulating a host neutrophil response during the course of an oncolytic infection. This may for example be carried out so as to augment the secondary neutrophil response, for example to enhance neutrophil mediated inflammation in the tumour. Again, this neutrophil response may be orchestrated so that it results in apoptotic killing of tumour cells.

In some embodiments, the oncolytic virus may mediate expression of an agent, such as a neutrophil modulating protein, that modulates the host neutrophil response. In alternative embodiments, the oncolytic virus may mediate expression of a neutrophil suppressing agent, such as a protein, that suppresses the host neutrophil response. In further alternative embodiments, the oncolytic virus may mediate expression of an agent, such as a neutrophil stimulating protein, that stimulates the host neutrophil response. An oncolytic virus may of course be constructed so as to mediate the expression of one or more of these activities at selected stages of the infective cycle, for example to combine two or more of these alternative effects.

In alternative embodiments, an effective amount of a neutrophil modulating agent may be administered to a host to modulate the host neutrophil response. An agent may for example suppress the host neutrophil response, release the suppression of the response, or stimulate the host neutrophil response. In the context of the invention, this modulation of the host neutrophil response includes steps taken to modulate an effect or symptom of the host neutrophil response, so as to suppress the effect or symptom of the host neutrophil response, release the suppression of the effect or symptom of the response, or stimulate the effect or symptom of the host neutrophil response. The effect or symptom of the host neutrophil response may for example be blood clot formation in the tumour vasculature.

In alternative embodiments, neutrophil suppressing agents may for example be selected from the following: neutrophil inhibitory factor (NIF, a glycoprotein isolated from a nematode, particularly that of the genus, Ancylostoma, as disclosed in EP 731709, U.S. Pat. No. 5,709,141, U.S. Pat. No. 5,747,296, U.S. Pat. No. 5,789,178, U.S. Pat. No. 5,919,900, U.S. Pat. No. 6,756,211, U.S. Pat. No. 6,818,616, U.S. Pat. No. 6,962,795, WO 1993/023063, and WO 1994/014973); glucosamine salts (as disclosed in EP 1238669, US 2002/128230, and U.S. Pat. No. 6,627,621); agonists of a neutrophil-secreted matrix metalloproteinase (MMP), such as MMP-9 (as disclosed in US 2002/159971 and WO 2002/066057); peptides derived from the amino acid sequence of neutrophil activating factor (NAF) that antagonise native NAF (U.S. Pat. No. 5,079,228); antibodies capable of binding to the CD11b subunit of the neutrophil integrin Mo1 (CD11b/CD18, as disclosed in WO 1992/005796); intraperitoneal administration of a monoclonal anti-granulocyte antibody (Tazawa et al. (2003); a small molecule selectin antagonist (Onai et al., 2003); inhibitors of a chemokine ligand/receptor complex, particularly CXCR2L/CXCR2 (Londhe et al., 2005); thalidomide (Yasui et al., 2005); tamoxifen (Grigoryants et al., 2005); a cysteinyl-leukotriene receptor antagonist (Benjamim et al., 2005); a platelet activating factor-receptor antagonist (Souza et al., 2003); IL-6, or IL-6 and TGFβ (U.S. Pat. No. 5,300,292); diethylmaleate (DEM), phorone, buthionine-sulfoximine (BSO), glutathione depleting diethylmaleate (DEM) mimetics, glutathione depleting phorone mimetics and glutathione depleting buthionine sulfoximine (BSO) mimetics (U.S. Pat. No. 5,994,402); peptides having the formula f-Met-Leu-X, where X is selected from the group consisting of Tyr, Tyr-Phe, Phe-Phe and Phe-Tyr (U.S. Pat. No. 6,462,020); recombinant proteins having the I-Domain from the human leukocyte beta2-integrin Mac-1 (WO 1995/029243); and decoy receptors for CXCR2 ligands, such as the Duffy antigen receptor for chemokines (DARC). In alternative embodiments, neutrophil suppressing agents may for example be anti-neutrophil antibodies, such as CAMPATH (anti CD52), anti-integrin antibodies, myelosuppressive chemotherapeutics (such as cyclophosphamide, and anthracycline) or anti-inflammatories such as the COX inhibitors, ASA, ibuprofen, or naproxyn.

In alternative embodiments, a neutrophil stimulating agent may, for example, be selected from the following: neutrophil-activating immune response modifier (IRM) and/or a toll-like receptor (TLR)-8-selective agonist (as disclosed in US 2005/96259 and WO 2005/041891); neutrophil-activating factor and structurally or functionally related peptides (NAF, as disclosed in U.S. Pat. No. 6,383,479, WO1989/004836, U.S. Pat. No. 5,759,533, WO 2001/066734 and WO 1990/006321); a neutrophil-activating polypeptide isolated from human mononuclear cells that has a molecular weight of 10 kDa (as disclosed in WO 1989/004325); ENA-78 (EP 538030); medium-chain fatty acids, glycerides, and analogues (US 2004/147599 and WO 2002/083120); proteins S100A8, S100A9, S100A12 or S100A8/A9 (WO 2004/084928); and compositions of one or more of GM-CSF, interferon-γ, tumor necrosis factor (TNF)-α, PAF (Grote et al., 2003; Jablonska et al., 2002; McClenahan et al., 2000), interleukin-8 (IL-8/CXCL1 homologues, such as Il-8((3-73))K11R; Li and Gordon, 2001) and chemotactic neutrophil receptor ligands (such as agonists of IL-8 receptors CXCR1 and CXCR2).

In some embodiments, an initial stage of neutrophil response suppression may be followed by a release of that suppresion. For example, an oncolytic virus may express a neutrophil stimulating agent, and the effect of that agent may be counteracted during the initial neutrophil response. For example an oncolytic virus may be engineered to express the peptide neutrophil stimulator ENA-78 (EP 538030). The effect of this neutrophil stimulator may be counteracted during the initial neutrophil response by an inhibitor of ENA-78 (as disclosed in U.S. Pat. No. 5,401,651 and U.S. Pat. No. 5,591,718).

In alternative embodiments, one or more strains of an oncolytic virus may be used in methods of the invention, simultaneously or successively. A virus may for example be selected from the group consisting of: adenovirus; reovirus; herpes simplex virus, such as HSV1; Newcastle disease virus; vaccinia virus; Coxsackievirus; measles virus; vesicular stomatitis virus (VSV); influenza virus; myxoma virus; Rhabdovirus, picornavirus.

In alternative embodiments, the invention may involve administering to a host a chemotherapeutic agent to augment killing of tumour cells during the secondary neutrophil response, such as chemotherapeutic agents that preferentially kills hypoxic tumour tissues. In alternative embodiments, the chemotherapeutic agent may for example be one or more of the following: dihydropyrimido-quinoxalines and dihydropyrimido-pyridopyrazines; quinoxaline or pyridopyrazine derivatives; 1,2-dihydro-8-piperazinyl-4-phenylimidazopyridopyrazine oxides and 1,2-dihydro-8-piperazinyl-4-phenylimidazo quinoxaline oxides; nitrophenyl mustard and nitrophenylaziridine alcohols, and their corresponding phosphates; anthraquinone compounds (as disclosed in WO 2005/061453); ligands based on alkylene amine oxime particularly butylene amine oxime ring structures, and radiometal complexes thereof (as disclosed in WO 1995/004552); 1,2,4 benzotriazine 1,4 dioxide compounds (WO 2005/082867); or nitro-substituted aromatic or hetero-aromatic compounds (EP 319329). In accordance with the illustrated effects herein of C. Novyi used in conjunction with VSV, alternative aspects of the invention involve the use of anaerobic bacteria as agents that preferentially kill hypoxic tumour tissues.

In alternative embodiments, the invention may be used to treat cancers, such as cancers characterized by the presence of solid tumours. These cancers may for example include both primary and metastatic solid tumors, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). In some aspects, methods and compositions of the invention may also be useful in treating solid tumors arising from hematopoietic malignancies such as leukemias (i.e. chloromas, plasmacytomas and the plaques and tumors of mycosis fungoides and cutaneous T-cell lymphoma/leukemia) and lymphomas (both Hodgkin's and non-Hodgkin's lymphomas).

In alternative embodiments, the oncolytic virus may be administered to the host systemically, such as intravenously, or intratumorally to infect the tumour. The oncolytic virus and a neutrophil modulating agent may for example be co-administered. Alternative hosts amenable to treatments in accordance with the invention may include animals, mammals and humans.

In alternative embodiments, the duration of neutrophil response suppression may be varied. From a time point commenced at the onset of oncolytic infection, suppression may for example be carried out for a period ranging from 1 hour, to 7 days, for example for 1, 2, 3, 4, 5, 6, 7, 8, 9, 10, 11, 12, 13, 14, 15, 16, 17, 18, 19, 20, 21, 22 or 23 hours; or for 1, 2, 3, 4, 5, 6 or 7 days.

In one aspect, in accordance with the methods of the invention, the invention provides for the use of one or more neutrophil modulating agents to modulate a neutrophil response to a solid tumour in a host during an oncolytic virus infection of the tumour, to increase the initial infectivity of the oncolytic virus in the solid tumour and to subsequently enhance neutrophil mediated inflammation in the tumour that results in apoptotic killing of tumour cells. In alternative aspects, the invention provides for the use of a neutrophil suppressing agent to increase the infectivity of an oncolytic virus in a solid tumour in a host. In a further aspect, the invention provides for the use of a neutrophil stimulating agent to release from a suppressed state a neutrophil response to a solid tumour infected with an oncolytic virus in a host.

In an alternative aspect, the invention involves the use of an anti-clotting agent to enhance viral infectivity in a tumour in an oncolytic therapy. In selected embodiments, the invention accordingly provides treatments which attenuate or ameliorate coagulation that attends a host neutrophil response. An anti-clotting agents may accordingly be used to formulate a medicament for enhancing viral infectivity in a tumour in an oncolytic therapy. Anti-clotting agents may for example be thrombolytic agents, fibrinolytic agents or anticoagulants.

### BRIEF DESCRIPTION OF THE DRAWINGS

FIG. 1: Illustrates evidence that neutrophil depletion results in inhibition of tumour growth. The graph plots tumour volume on the Y axis against days on the X axis. To obtain the data, 6-8 week old Balb/C mice were injected subcutaneously with 3×10⁵ CT-26 cells. Subcutaneous tumours were allowed to develop for 14 days. Mice were given intraperitoneal injections of 100 ul 50:50 rat serum:PBS control or 100 ug anti-Ly6G antibody every other day, starting day −1. All mice were treated with 5×10⁸ pfu D51 VSV GFP-firefly luciferase on day 0 and mouse that was treated twice received second dose on day 2. Tumour dimensions were measured with a caliper and tumour volume was calculated as tumour length²*tumour width/2.

FIG. 2: illustrates the augmentation of tumour necrosis (apoptosis) using treatments that target tumour tissues that have become hypoxic as the result of a neutrophil mediated response to an oncolytic infection of the tumour. The graphs show the response of CT26 tumour bearing mice treated with VSV or C. Novyi, alone or in combination. As discussed in Example 2, Balb/c mice with CT26 subcutaneous tumours were treated intravenously with one dose of 10⁷ pfu D51-VSV plus 10⁴ C. Novyi spores (panel A), or with 10⁴ C. Novyi spores alone (panel B), or with 10⁷ pfu D51-VSV alone (panel C). Survival rates of treated mice are shown in panel D. The combination of VSV and C. Novyi demonstrated superior tumour responses than either agent alone.

FIG. 3 illustrates embodiments in which anti-clotting treatments prevent tumor vascular shutdown and promote virus spread, as evidenced by visualization of fluorescent microspheres and virus distribution (immunohistochemistry of VSV antigens) in a murine CT26 tumour model.

FIG. 4 is a schematic showing a protocol that illustrates the use of heparin to modulate tumour perfusion during oncolytic viral therapy. The results derived from this protocol are illustrated in FIGS. 5 through 14.

FIG. 5 shows comparative immunohistochemistry of CT-26 tumour sections, illustrating fibrin deposition in tumour vessels.

FIG. 6 is a graph illustrating the time frame of the oncolytic clot forming effect.

FIG. 7 shows immunohistochemistry of CT-26 tumour sections 24 hours post virus (VSV) treatment, illustrating fibrin distribution.

FIG. 8, in contrast to FIG. 7, shows immunohistochemistry of CT-26 tumour sections 24 hours post heparin and virus (VSV) treatment, illustrating that if heparin is included with the virus, it blocks the deposition of fibrin and therefore clot formation.

FIG. 9 is a panel of tumour section micrographs from 24 hours post VSV infection in the CT-26 tumour model, illustrating by immunohistochemistry VSV distribution (infection), and active caspase 3 (apoptosis); and illustrating by scanning of fluorescent microspheres the degree of perfusion.

FIGS. 10 and 11 are panels of tumour section micrographs from 24 hours post VSV infection with heparin treatment in the CT-26 tumour model, illustrating by immunohistochemistry VSV distribution (infection), and active caspase 3 (apoptosis); and illustrating by scanning of fluorescent microspheres the degree of perfusion.

FIGS. 12 and 13 are panels of tumour section micrographs from 5 days post VSV infection with heparin treatment in the CT-26 tumour model, illustrating by immunohistochemistry VSV distribution (infection), and active caspase 3 (apoptosis); and illustrating by scanning of fluorescent microspheres the degree of perfusion.

### DETAILED DESCRIPTION

In one aspect, the invention relates to the demonstration that an unrestrained host neutrophil response may attenuate an oncolytic infection in a tumour. In accordance with the following Examples, suppression of a neutrophil response, for example by depletion of neutrophils, prior to oncolytic infection, permits more extensive spread of the virus throughout the tumour.

In an alternative aspect, the invention relates to the demonstration that an appropriate host neutrophil response may augment the killing of tumour cells in the course of an oncolytic infection. In various embodiments of the invention, a significant portion of the in vivo tumour killing activity of oncolytic viruses is not caused by direct cell lysis, but rather by indirect or “bystander” killing. An initial neutrophil response, following even limited virus infection, results in a loss of blood flow to the interior of the tumour, and this correlates with massive induction of cellular apoptosis within the tumour.

Accordingly, in aspects that combine the forgoing effects, the present invention involves deferring the targeted recruitment of neutrophils to infected tumour beds, or deferring a symptom or effect of this recruitment (such as clotting or vascular shut down in tumour vasculature) to facilitate viral infection at an initial stage of therapy, and to enhance cancer cell “bystander” killing in a later stage of therapy.

In alternative embodiments, the invention may involve suppressing a host neutrophil response to an oncolytic infection. The suppression may for example be carried out so that the host has a suppressed neutrophil condition during the initial neutrophil response to the oncolytic infection. As demonstrated herein, this suppression may be modulated so as to increase the number of tumour cells infected with the oncolytic virus during the initial neutrophil response, compared to the number that would be infected in the absence of the step of suppressing the host neutrophil response. Depletion of neutrophils by chemotherapy or by antibody mediated depletion is contemplated. Neutrophil suppressing agents may for example be selected from the following: neutrophil inhibitory factor (NIF, a glycoprotein isolated from a nematode, particularly that of the genus, Ancylostoma, as disclosed in EP 731709, U.S. Pat. No. 5,709,141, U.S. Pat. No. 5,747,296, U.S. Pat. No. 5,789,178, U.S. Pat. No. 5,919,900, U.S. Pat. No. 6,756,211, U.S. Pat. No. 6,818,616, U.S. Pat. No. 6,962,795, WO 1993/023063, and WO 1994/014973); glucosamine salts (as disclosed in EP 1238669, US 2002/128230, and U.S. Pat. No. 6,627,621); agonists of a neutrophil-secreted matrix metalloproteinase (MMP), such as MMP-9 (as disclosed in US 2002/159971 and WO 2002/066057); peptides derived from the amino acid sequence of neutrophil activating factor (NAF) that antagonise native NAF (U.S. Pat. No. 5,079,228); antibodies capable of binding to the CD11b subunit of the neutrophil integrin Mo1 (CD11b/CD18, as disclosed in WO 1992/005796); intraperitoneal administration of a monoclonal anti-granulocyte antibody (Tazawa et al. (2003); a small molecule selectin antagonist (Onai et al., 2003); inhibitors of a chemokine ligand/receptor complex, particularly CXCR2L/CXCR2 (Londhe et al., 2005); thalidomide (Yasui et al., 2005); tamoxifen (Grigoryants et al., 2005); a cysteinyl-leukotriene receptor antagonist (Benjamim et al., 2005); a platelet activating factor-receptor antagonist (Souza et al., 2003); IL-6, or IL-6 and TGFβ (U.S. Pat. No. 5,300,292); diethylmaleate (DEM), phorone, buthionine-sulfoximine (BSO), glutathione depleting diethylmaleate (DEM) mimetics, glutathione depleting phorone mimetics and glutathione depleting buthionine sulfoximine (BSO) mimetics (U.S. Pat. No. 5,994,402); peptides having the formula f-Met-Leu-X, where X is selected from the group consisting of Tyr, Tyr-Phe, Phe-Phe and Phe-Tyr (U.S. Pat. No. 6,462,020); recombinant proteins having the I-Domain from the human leukocyte beta2-integrin Mac-1 (WO 1995/029243); small molecules such as N,N′-diarylureas or repertaxin (Widdowson et al., 2004; Souza et al., 2004) or peptides such as CXCL8((3-73))K11R/G31P (Li et al., 2002); a neutrophil elastase inhibitor, such as N-[2-[4-(2,2-dimethyl propionyloxy)phenylsulfonylamino]benzoyl]aminoacetic acid (Takai et al., 2005); or neutralizing antibodies to IL-8 (Huang et al., (2002); Mian et al. (2003).

In alternative embodiments, an oncolytic virus is engineered to suppress a neutrophil response. For example, by expressing mediating expression of a peptide neutrophil suppressor. In one embodiment, an oncolytic virus may mediate expression of a decoy receptor for CXCL1 (Addison et al. BMC Cancer. (2004) 4:28.) or a peptide that inhibits CXCR2 binding to CXCL1 (Li et al Vet Immunopathol (2002) 90:65-77) in the tumour environment, so neutrophils are not recruited to the tumour, in this way the oncolytic virus mediates a suppressed neutrophil condition in the host during the initial neutrophil response, so as to increase the number of tumour cells infected with the virus during the initial neutrophil response compared to the number that would be infected in the absence of the step of suppressing the host neutrophil response. Under these conditions, an oncolytic virus may be able to spread more effectively throughout a tumour. In selected embodiments, the neutrophil response of the host is modulated so that the systemic neutrophil activity in the host is not affected as significantly as the specific neutrophil response to the infected tumour, in this way, modulation of the neutrophil response may take place so that the patient is not immunocompromised. In alternative aspects, the invention may involve localized neutrophil inhibition at the tumour site, which may for example be achieved by engineering an oncolytic virus to express a neutrophil inhibiting agent.

In various aspects, the invention involves steps of stimulating a neutrophil response, for example by stimulating a response to a tumour infected with an oncolytic virus. As outlined in the Background section, a significant number of compositions have been described that are useful for neutrophil stimulation, and those compositions and methods may be adopted in alternative embodiments of the present invention. For example, a virus, such as an oncolytic virus that expresses CXCL1 may be used to enhance neutrophil recruitment. This may be particularly advantageous in embodiments in which tumours are treated that do not express CXCL1 upon oncolytic infection (such as 4T1 (CT26) tumours as opposed to CT26 (4T1) tumours). In alternative embodiments, treatments of the invention may infect a tumour first with an oncolytic virus that does not express a neutrophil stimulator, such as CXCL1, so that the neutrophil response to the tumour is suppressed, and follow this first stage of oncolytic infection with a second virus that expresses CXCL1 so as to stimulate the neutrophil response to the infected tumour.

In alternative embodiments, a neutrophil stimulating agent may, for example, be selected from the following: neutrophil-activating immune response modifier (IRM) and/or a toll-like receptor (TLR)-8-selective agonist (as disclosed in US 2005/96259 and WO 2005/041891); neutrophil-activating factor and structurally or functionally related peptides (NAF, as disclosed in U.S. Pat. No. 6,383,479, WO1989/004836, U.S. Pat. No. 5,759,533, WO 2001/066734 and WO 1990/006321); a neutrophil-activating polypeptide isolated from human mononuclear cells that has a molecular weight of 10 kDa (as disclosed in WO 1989/004325); ENA-78 (EP 538030); medium-chain fatty acids, glycerides, and analogues (US 2004/147599 and WO 2002/083120); proteins S100A8, S100A9, S100A12 or S100A8/A9 (WO 2004/084928); compositions of one or more of GM-CSF, interferon-γ, tumor necrosis factor (TNF)-α and platelet activating factor PAF (Grote et al., 2003; Jablonska at al., 2002; McClenahan et al., 2000); a virus expressing a protein, such as GM-CSF, may be used to stimulate neutrophils (Jablonska et al., 2002; Grote et al., 2003); interleukin-8 (IL-8/CXCL1 homologues, such as 11-8((3-73))K11 R; Li and Gordon, 2001) and other chemotactic neutrophil receptor ligands (such as agonists of IL-8 receptors CXCR1 and CXCR2).

In one aspect, the present invention involves the recognition that a number of cytokines are up-regulated in the course a robust neutrophil response to a tumour infected with an oncolytic virus. Accordingly, these cytokines, or viruses expressing these cytokines, may be used, by administration or co-administration with an oncolytic virus, to augment the apoptotic tumour cell killing that takes place in a host neutrophil response to the infected toumour. The cytokines that have been identified as of use in this aspect of the invention include Gro alpha, ENA-78, MCP-1, IP-10, MIP2, Interferon-β, M-CSF, RANTES, MIP-1beta, MIP-1alpha, calgranulin A, calgranulin B, or combinations thereof.

### Cancers and Related Indications

In various aspects, the invention provides compositions and methods for treating cancers, and related conditions treatment of benign, inoperable mass. For example the invention may involve the treatment of cancers characterized by the presence of solid tumours, including both primary and metastatic solid tumors, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). In some aspects, methods and compositions of the invention may also be useful in treating solid tumors arising from hematopoietic malignancies such as leukemias (i.e. chloromas, plasmacytomas and the plaques and tumors of mycosis fungoides and cutaneous T-cell lymphoma/leukemia) and lymphomas (both Hodgkin's and non-Hodgkin's lymphomas). In addition, aspects of the invention may be useful in the prevention of metastases from the tumors described above either when used alone or in combination with additional therapeutic approaches, such as radiotherapy or chemotherapy.

### Therapeutic Formulations

In one aspect, the invention involves administration (including co-administration) of therapeutic compounds or compositions, such as an oncolytic virus or agents that are effective to modulate a neutrophil response in a host. In various embodiments, such agents may be used therapeutically in formulations or medicaments. Accordingly, the invention provides therapeutic compositions comprising active agents, including agents that stimulate a neutrophil response and/or agents that suppress a neutrophil response, and pharmacologically acceptable excipients or carriers.

An effective amount of an agent of the invention will generally be a therapeutically effective amount. A “therapeutically effective amount” generally refers to an amount effective, at dosages and for periods of time necessary, to achieve the desired therapeutic result, such as modulation of a neutrophil response. A therapeutically effective amount a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects.

In particular embodiments, a preferred range for therapeutically effective amounts of a neutrophil modulating agent may be 0.1 nM to 0.1 M, 0.1 nM to 0.05 M, 0.05 nM to15 uM or 0.01 nM to 10 uM. Alternatively, total daily doses may range from about 0.001 mg/kg to about 1 mg/kg of patients body mass. Dosage values may vary with the severity of the condition to be alleviated. It is to be further understood that for any particular subject, specific dosage regimens should be adjusted over time according to the individual need and the professional judgement of the person administering or supervising the administration of the compositions, and that dosage ranges set forth herein are exemplary only and are not intended to limit the scope or practice of the methods of the invention.

A “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, active agents of the invention may be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active agent in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In accordance with another aspect of the invention, therapeutic agents of the present invention, such as neutrophil response modulating agents, may be provided in containers having labels that provide instructions for use of, or to indicate the contents as, neutrophil response modulating compounds, such as compounds to suppress or stimulate a neutrophil response, for treating cancers, such as cancers characterized by solid tumours.

Use of the present invention to treat or prevent a disease condition as disclosed herein, including prevention of further disease progression, may be conducted in subjects diagnosed or otherwise determined to be afflicted or at risk of developing the condition. In some embodiments, for oncolytic therapy, patients may be characterized as having adequate bone marrow function (for example defined as a peripheral absolute granulocyte count of >2,000/mm³ and a platelet count of 100,000/mm³), adequate liver function (for example, bilirubin<1.5 mg/dl) and adequate renal function (for example, creatinine<1.5 mg/dl).

Routes of administration for agents of the invention may vary, and may for example include intradermal, transdermal, parenteral, intravenous, intramuscular, intranasal, subcutaneous, regional, percutaneous, intratracheal, intraperitoneal, intraarterial, intravesical, intratumoral, inhalation, perfusion, lavage, direct injection, and oral administration and formulation.

Intratumoral injection, or injection into the tumor vasculature is contemplated for discrete, solid, accessible tumors. Local, regional or systemic administration also may be appropriate. For tumors of >4 cm, the volume to be administered may for example be about 4 to 10 ml, while for tumors of <4 cm, a volume of about 1 to 3 ml may be used. Multiple injections may be delivered as single dose, for example in about 0.1 to about 0.5 ml volumes. Viral particles may be administered in multiple injections to a tumor, for example spaced at approximately 1 cm intervals.

Methods of the present invention may be used preoperatively, for example to render an inoperable tumor subject to resection. Alternatively, the present invention may be used at the time of surgery, and/or thereafter, to treat residual or metastatic disease. For example, a resected tumor bed may be injected or perfused with a formulation comprising an oncolytic virus. The perfusion may for example be continued post-resection, for example, by leaving a catheter implanted at the site of the surgery. Periodic post-surgical treatment may also be useful.

Continuous administration of agents of the invention may be applied, where appropriate, for example, where a tumor is excised and the tumor bed is treated to eliminate residual, microscopic disease. Continuous perfusion may for example take place for a period from about 1 to 2 hours, to about 2 to 6 hours, to about 6 to 12 hours, to about 12 to 24 hours, to about 1 to 2 days, to about 1 to 2 weeks or longer following the initiation of treatment. Generally, the dose of the therapeutic agent via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatments of the invention may include various “unit doses.” A unit dose is defined as containing a predetermined-quantity of the therapeutic composition. A unit dose need not be administered as a single injection but may comprise continuous infusion over a set period of time. Unit dose of the present invention may conveniently be described in terms of plaque forming units (pfu) for a viral construct. Unit doses range from 10³, 10⁴, 10⁵, 10⁶, 10⁷, 10⁸, 10⁹, 10¹⁰, 10¹¹, 10¹², 10¹³ pfu and higher. Alternatively, depending on the kind of virus and the titer attainable, one may deliver 1 to 100, 10 to 50, 100 to 1000, or up to about 10⁴, 10⁵, 10⁶, 10⁷, 10⁸, 10⁹, 10¹⁰, 10¹¹, 10¹², 10¹³, 10¹⁴, or 10¹⁵ or higher infectious viral particles (vp) to the patient or to the patient's cells.

### Example 1

The present Example discloses aspects of the tumour microenvironment at a short timepoint post infection with an oncolytic virus. In this example, a dose of VSV was administered to a syngeneic Balb/C mouse with established subcutaneous CT-26 tumours. The results indicate that a rapid immune response creates a barrier to VSV spread in vivo.

CT-26 tumour bearing mice were treated once intravenously with VSV and sacrificed 24 hours later. Sections were prepared for immunohistochemical analysis for VSV and active caspase 3 as a marker of apoptosis. Unexpectedly, VSV staining was restricted to a limited number of peripheral tumour sites by 24 h, yet extensive apoptosis is detected through much of the tumour, as evidenced by active of caspase 3 staining. Apoptosis was confirmed in VSV treated tumours by TUNEL staining. Hematoxylin & eosin staining was performed on CT-26 tumours removed 5 days post infection and revealed extensive apoptosis by cell morphology. In contrast, untreated tumours of matched size or tumours from animals treated with UV virus showed very little apoptosis.

These results demonstrate that there is significant killing of cells in treated tumours that occurs without direct infection, a phenomena that may be referred to as “bystander killing”. Bystander killing was also observed when tumours were treated with an oncolytic version of vaccinia virus. Extensive apoptosis induced by vaccinia infection was maximal about 120 hours following treatment.

The massive cell death occurring in the absence of direct virus infection is accompanied by alterations in tumour blood flow. As evidence of this, tumour bearing mice were treated with either VSV or vaccinia virus and then at various time points, fluorescent microspheres were given intravenously and then animals sacrificed and tumours excised 5 minutes later. Since the microspheres access any areas of organs (and tumour) that are supplied with blood, they are a useful tool to measure perfusion of the tumour. Fluorescence was detected on 10 um frozen sections by microarray scanner. Control tumours treated with UV inactivated VSV contain uniformly distributed microspheres. However, 24 h post infection with VSV or 120 h post vaccinia virus administration, much of the tumour is not accessed by microspheres, whereas adjacent normal muscle tissue remains uniformly perfused.

The foregoing results indicate that limited virus replication within the tumour leads to a reduction in blood flow and consequently acute hypoxia causing massive cellular apoptosis in uninfected tumour regions.

Transcriptional profiling of infected tumours identifies particular gene products, known to be involved in innate cellular immunity, that are locally produced during oncolytic virus therapy, including the following: COX2, prostaglandin-endoperoxide synthetase 2; IL-15, interleukin 15; IFN-β, interferon beta, fibroblast complement component 3; M-CSF, Colony stimulating factor 1 (macrophage); IL-6, interleukin 6; MCP1, chemokine (C-X-C motif) ligand 2; CXCL5, chemokine (C-X-C motif) ligand 5; CXCL11, chemokine (C-X-C motif) ligand 11; IP10, chemokine (C-X-C motif) ligand 10; KC, chemokine (C-X-C motif) ligand 1; MCP3, chemokine (C-C motif) ligand 7; RANTES, chemokine (C-C motif) ligand 5; MIP1β, chemokine (C-C motif) ligand 4; MIP1α, chemokine (C-C motif) ligand 3; MCP1, chemokine (C-C motif) ligand 2; S100 calcium binding protein A9 (calgranulin B); S100 calcium binding protein A8 (calgranulin A); chemokine (C-C motif) receptor-like 2; VCAM-1, Vacular cell adhesion molecule 1; Selectin, endothelial cell; ICAM, intercellular adhesion molecule. Many of the genes that were upregulated in response to VSV infection are NF-κB responsive, including the neutrophil chemoattractants CXCL1, CXCL2 and CXCL5. Accordingly, this transcript profiling of infected tumours shows that virus infection results in a dramatic transcriptional activation of pro-inflammatory genes including the neutrophil chemoattractant CXCL1. Immunohistochemical examination of infected tumours revealed infiltration by neutrophils correlating with CXCL1 induction.

To demonstrate that neutrophils are involved in the bystander killing of tumours, we depleted tumour bearing mice of neutrophils prior to intravenous therapy with VSV. Tumours from VSV treated, neutrophil depleted mice did not display the hallmarks of the bystander effect in that they were well perfused and did not have large regions of apoptosis. Also, in the neutrophil depleted animals, VSV spread more efficiently, indicating that the neutrophil mediated bystander effect is involved in preventing virus spread within the tumour. In neutrophil depleted animals, active caspase 3 staining is more closely associated with sites of virus infection, again indicating the absence of bystander killing.

To illustrate that neutrophil suppression affects the kinetics of viral replication, VSV infection was analyzed in vivo using an IVIS™ system (Xenogen Corporation) and VSV expressing firefly luciferase. Tumour-bearing mice were given i.p. injections of anti-Ly6G antibody every other day (starting 1 day prior to VSV infection). Mice were imaged every day for 5 days following infection with virus. VSV replication was detected until day 3 post infection in mice that received i.p. injections of the control non-immune rat serum, while mice that were depleted of neutrophils with the anti-Ly6G antibody demonstrated tumour specific viral replication until 5 days post infection.

The present Example demonstrates that changes in gene expression triggered shortly after virus infection result in innate immune cell recruitment to an infected tumour, disruption of tumour blood flow and extensive localized tumour cell death. The bystanding killing phenomenon is caused by diverse viral agents, by VSV, a rapidly replicating, immune stimulatory agent, and also by vaccinia virus, which is a more complex virus with a longer replication cycle and a greater ability to manipulate host cell gene expression.

This Example illustrates that viral replication is more uniform throughout the tumour and persists longer, with neutrophil suppression or depletion. There is evidence that this is at least in part due to the fact that a neutrophil response to an infected tumour leads to vascular shutdown in the tumour. The results obtained from IVIS™ visualization of virus replication indicate that sustained systemic granulocyte depletion does not increase the systemic virulence of VSV. This is evident from the fact that viral replication is exclusively detected in the tumour as of one day post infection.

### Human Model of the Bystander Effect:

SW620 human colon carcinoma cells were injected subcutanesouly into CD1 nude mice and tumors were allowed to grow for 30-40 days. Once tumours were palpable, mice were treated with 5×10⁸ pfu D51 VSV GFP and sacrificed 24 h later. Tumors removed from treated mice demonstrated limited VSV replication with extensive apoptosis in large uninfected areas of the tumor. Prior to sacrifice, mice were infused with fluorescent microspheres and as with murine CT-26 tumors, microsphere distribution was limited to small areas in the rim of the tumor, indicating vascular shutdown of the tumor core. In contrast, uninfected SW620 tumours demonstrate more uniform distribution of microspheres.

Inducing the Bystander Effect with a Neutrophil Stimulating Virus

Orthotopic 4T1 mouse mammary gland tumors were established in the fat pad of Balb/C mice. Treatment with PBS, recombinant GM-CSF or 5×10⁸ pfu D51 VSV GFP alone does not result in vascular shutdown and bystander killing in this model. However, treatment of 4T1 tumour bearing mice with D51 VSV expressing GM-CSF resulted in increased apoptosis of uninfected tumour cells as well as a shutdown of blood flow to the tumor core.

### Methods

### Viruses

The Indiana serotype of VSV was used in this Example, and was propagated in Vero cells (ATCC). Δ51 VSV expressing GFP is a recombinant interferon inducing mutant of the HR strain of wild-type VSV Indiana. Double deleted Vaccinia virus (thymidine kinase and vaccinia growth factor deleted) was prepared on Vero cells.

### Cells

CT26 (murine colon adenocarcinoma), 4T1 (murine mammary epithelial tumor line) and SW620 (human colon carcinoma) cells were purchased from ATCC and cultured in HyQ Dulbecco's Modified Eagle Medium (High glucose) with L-glutamine and sodium pyruvate (HyClone) supplemented with a 3:1 mixture of bovine serum (Medicorp): fetal calf serum (CanSera), made up to 10%. Cells were incubated at 37° C. in 5% CO₂. Subconfluent CT26 cells were harvested by trypsinization, pelleted, resuspended in PBS and assessed for viability by trypan blue staining.

### Tumor Models

Female 6-8 week old Balb/C mice were obtained from Charles River Laboratories. Syngeneic subcutaneous tumors were established by injection of 3×10⁵ cells in 100 ul PBS in the left and right hind flanks. When tumors reached a palpable size within 10 to 14 days, mice were treated with virus by tail vein injection. Mice were sacrificed at the indicated timepoints by cervical dislocation and tumors and other organs were frozen in Shandon Cryomatrix freezing medium (ThermoElectron Corporation) on dry ice. Tissues were stored at −80° C. until sectioning in a cryostat (Microm HM500 OM). Five micron sections were cut and adhered to Fischer Superfrost Plus slides and stored at −80° C. until processing.

### H & E Staining and Immunohistochemistry

Immunohistochemistry was performed using the Vectastain ABC kit for rabbit primary antibodies (Vector Labs), according to instructions provided. Unless otherwise indicated, dilutions were made in PBS, incubations were carried out at room temperature and samples were washed several times with PBS between each step. Briefly, tissue sections were fixed in fresh 4% paraformaldehyde (20 min), quenched of endogenous peroxidase activity with 3% H₂O₂ (15 min) and then blocked with 1.5% normal goat serum (1 hour). Endogenous biotin was blocked with Avidin solution (15 min) then Biotin solution (15 min) using an Avidin Biotin Blocking kit (Vector Labs). Primary antibodies (all of rabbit origin) were employed as follows: VSV (gift of Earl Brown) at 1/5000 for 1 h, active caspase 3 (BD Pharmingen) at 1/500 for 1 h. Secondary antibody and ABC reagent provided with the Vectastain ABC kit were applied as instructed. Horseradish peroxidase (HRP) activity was visualized with a Diaminobenzene-HRP kit (KPL Biosciences), resulting in formation of a brown precipitate in positive areas. Nuclei were counterstained in hematoxylin. Slides were dehydrated in an ethanol/xylenes series and mounted according to standard protocols. For assessment of cell morphology, sections were stained with hematoxylin and eosin according to standard protocols. Whole tumor images were obtained with an Epson Perfection 2450 Photo Scanner while magnifications were captured using a Xeiss Axiophot HBO 50 microscope.

### Immunofluorescence and TUNEL Staining

Unless otherwise indicated, dilutions were made in PBS, incubations were carried out at room temperature and samples were washed several times with PBS between each step. Briefly, tissue sections were fixed in fresh 4% paraformaldehyde (20 min), blocked with 1.5% normal goat serum (30 min) and incublated with rabbit anti-serum raised against VSV (gift from Earl Brown) at 1/5000 dilution for 30 min. Then a Cy3 conjugated donkey anti-rabbit antibody (Jackson lmmunoresearch Laboratories) was applied (1/500) for 30 min. TUNEL staining was carried out according to manufacturer's instructions (In Situ Cell Death Detection Kit—FITC, Roche) with enzyme incubation at 37° C. for one hour. Nuclei were counterstained with Hoechst33242 (2.5 ug/ml) and slides were coverslipped in PBS/glycerol. Staining was visualized in a Zeiss Axiocam HRM Inverted fluorescent microscope and analyzed using Axiovision 4.0 software. Negative control sections were used to set exposures, which were kept constant throughout.

### Analysis of Tumor Perfusion

Mice were injected intravenously with 100 ul of a 50% solution of 100 nm diameter orange fluorescent microspheres (Molecular Probes). Five minutes later, animals were sacrificed and tumors immediately snap frozen as previously described. Tumor perfusion was analyzed by visualizing fluorescent microspheres in the vasculature of 10 um unfixed frozen sections using a ScanArray Express microarray scanner with a standard Cy3 laser (Packard Bioscience).

### Reverse Transcription and Q-PCR

CAT RNA was made by in vitro transcription with the RiboMAX™ Large Scale RNA Production Systems (Promega, Madison, Wis.) using the pCAT plasmid as template. Total tumor or cell derived RNA was reverse transcribed (1 or 2 μg RNA) with a spike of 5 ng of CAT RNA, an exogenous control used to quantitate and normalize for reverse transcription (RT) efficiency. Quantitative real-time PCR (qPCR) was performed in triplicate on all samples on the Roche LightCycler rapid thermal cycler system (according to the manufacturer's instructions) and using the FastStart DNA Master SYBR Green I kit (Roche Diagnostics, Laval, Canada). Standard curves were initially generated by standard dilutions and used to find absolute values for each reaction. All values obtained were normalized to CAT values to normalize the RT efficiency. Primers were designed with Primer3 software and 2 sets of primers were designed for each gene, each was tested and only the best was used. Primers were optimized for MgCl₂ for concentrations between 2 and 4 mM. All primer sets used are used at 3 mM, except for M-csf that is used at 4 mM. Primers used for qPCR are listed in Table 1 below, with left and right sequences and product size that result.

TABLE 1

| | | | Product |
|---|---|---|---|
| Gene Name | Left | Right | size |
| M-csf | ctggaaggaggatcagcaag | ccccacagaagaatccaatg | 246 |
| Cox2 | tcctcctggaacatggactc | ccccaaagatagcatctgga | 321 |
| C3 | ctgtgtgggtggatgtgaag | ttggtgcactcaagatctgc | 340 |
| CXCL1 | gctgggattcacctcaagaa | gtcagaagccagcgttcac | 231 |
| CXCL2 | tccagagcttgagtgtgacg | aggcacatcaggtacgatcc | 258 |
| II-6 | aacgatgatgcacttgcaga | ggaaattggggtaggaagga | 276 |
| IFN beta | ataagcagctccagctccaa | ctgtctgctggtggagttca | 267 |
| Vcam1 | gtggtgctgtcacaatgacc | acgtcagaacaaccgaatcc | 288 |
| E- | ccagtgcttctggacctttc | caagctaaagccctcattgc | 257 |
| selectin | | | |
| II-1beta | caggcaggcagtatcactca | agctcatatgggtccgacag | 249 |
| TNF | ccacatctccctccagaaaa | agggtctgggccatagaact | 259 |
| alpha | | | |
| CAT | gcgtgttacggtgaaaacct | gggcgaagaagttgtccata | 133 |

### In Vivo Neutrophil Depletion

Mice were injected i.p. with 7.5 ug purified anti-Ly6G rat monoclonal antibody, clone RB6-8C5 (BD Pharmingen) in order to systemically deplete granulocytes. 150 ul 50:50 non-immune rat serum:PBS was utilized as a negative control. 24 h later, mice were treated i.v. with 5×10⁸ pfu Δ51 VSV GFP, perfused with fluorescent microspheres 24 h later and sacrificed by cervical dislocation.

### In Vivo Imaging of Viral Replication

Mice were injected i.p. with 100 ug purfied anti-Ly6G rat monoclonal antibody, clone RB6-8C5 every other day in order to chronically deplete mice of granulocytes. 100 ul 50:50 non-immune rat serum:PBS was utilized as negative control. 24 h after the first administration of antibody, mice were treated i.v. with 5×10⁸ pfu Δ51 VSV GFP luciferase (firefly) and mice were imaged for luminescence starting 1 day post infection using the Xenogen IVIS® system. Identical scale bars were used on all images.

### Example 2

### Neutrophil Depletion Using Targeted Anti-Ly6G Antibody Abrogates Vascular Shutdown and Bystander Tumour Cell Apoptosis in VSV Treated Subcutaneous CT-26 Tumours and Results in Prolonged VSV Replication in the Tumour

In this Example, two imaging techniques provide corroborating evidence that suppression of a neutrophil response enhances oncolytic infection of a tumour.

### Tissue Sections

Tumour tissue cross sections were stained and visualized as follows. 6-8 week old Balb/C mice were injected subcutaneously with 3×10⁵ CT-26 cells. Subcutaneous tumours were allowed to develop for 14 days. Mice were given intraperitoneal injections of 150 ul 50:50 rat serum:PBS control or 7.5 ug anti-Ly6G antibody in PBS. 24 h later, mice were treated intravenously with 5×10⁸ pfu D51 VSV GFP. At 24 h after treatment with virus, mice were injected with 0.1 uM diameter fluorescent microspheres intravenously and sacrificed 5 min later. Tumours were excised, embedded in OCT medium and stored at −80C. 5 uM sections were prepared for immunohistochemistry (anti-rabbit secondary control, rabbit polyclonal anti-VSV 1:5000, rabbit monoclonal anti-active Caspase 3 1:500). Fluorescent microspheres were visualized in 10 uM sections with a ScanArray Express microarray scanner. The sections illustrated a pattern of staining that indicates that in the absence of neutrophil depletion (suppression), there is vascular shutdown, with infected and apoptotic regions generally confined to the periphery of the tumour. With neutrophil depletion, microspheres are more evenly distributed throughout the tumour, as are regions of oncolytic infection and apoptosis, confirming that suppression of a neutrophil response enhances oncolytic infection of a tumour.

### In Vivo Visualization

In vivo visualization of tumours in a mouse model was carried out as follows. 6-8 week old Balb/C mice were injected subcutaneously with 3×10⁵ CT-26 cells. Subcutaneous tumours were allowed to develop for 14 days. Mice were given intraperitoneal injections of 100 ul 50:50 rat serum:PBS control or 100 ug anti-Ly6G antibody every other day, starting day −1. All mice were treated with 5×10⁸ pfu D51 VSV GFP-firefly luciferase on day 0 and imaged with the IVIS system every day starting day 1. Scale normalized to day 1 anti-Ly6G+VSV (b). A time course of images from day 1 to day 4 illustrated a significantly more prolonged and robust oncolytic VSV infection in neutrophil depleted animals.

### Example 3

### Neutrophil Depletion Results in Inhibition of Tumour Growth

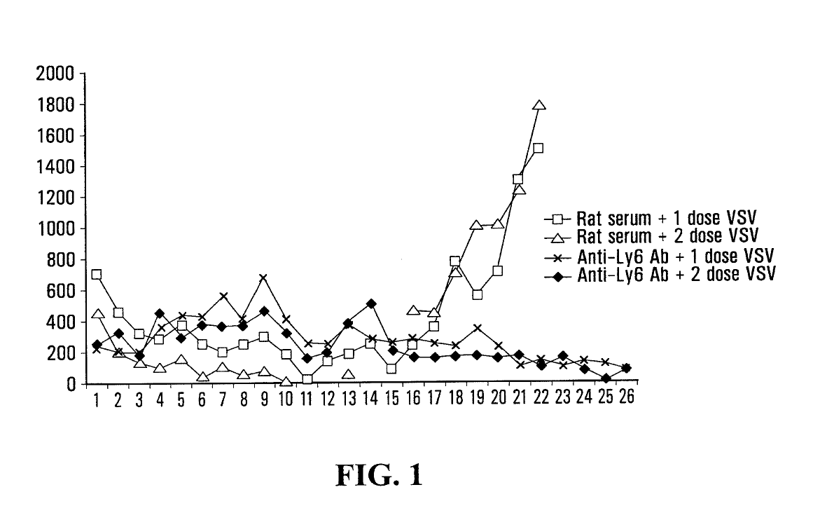

In this Example, 6-8 week old Balb/C mice were injected subcutaneously with 3×10⁵ CT-26 cells. Subcutaneous tumours were allowed to develop for 14 days. Mice were given intraperitoneal injections of 100 ul 50:50 rat serum:PBS control or 100 ug anti-Ly6G antibody every other day, starting day −1. All mice were treated with 5×10⁸ pfu D51 VSV GFP-firefly luciferase on day 0 and mouse that was treated twice received second dose on day 2. Tumour dimensions were measured with a caliper and tumour volume was calculated as tumour length²*tumour width/2. As illustrated in FIG. 1, neutrophil depletion results in inhibition of tumour growth. The graph plots tumour volume on the Y axis against days on the X axis, illustrating that neutrophil depletion results in inhibition of tumour growth.

### Example 4

### Exploiting the Bystander Effect

In some embodiments, the invention may involve treatments that capitalize on the recognition that a neutrophil response to a tumour infected by an oncolytic virus gives rise to vascular shutdown, and thereby creates hypoxic regions within infected tumours. A number of chemotherapeutic agents are available to specifically target hypoxic tumour tissues, for example: tirapazamine (Lunt et al., 2005). In alternative embodiments, methods of the invention may utilize biologics that attack hypoxic regions, such as anaerobic bacteria. This effect is demonstrated in this Example using C. Novyi.

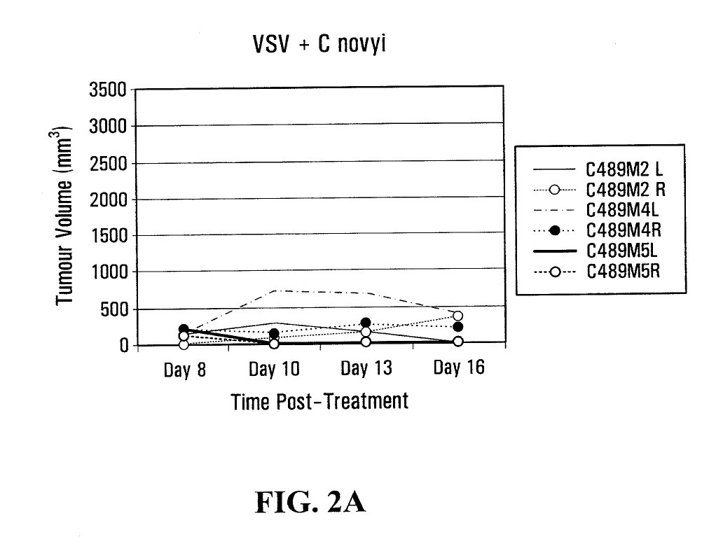

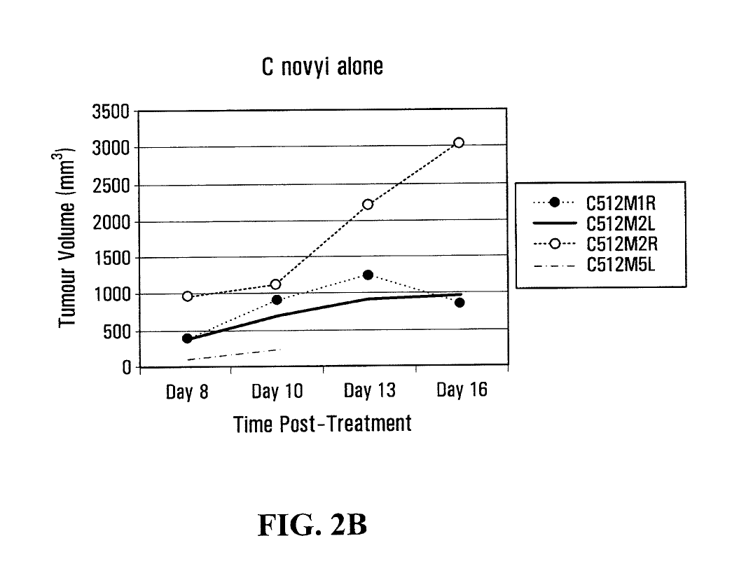

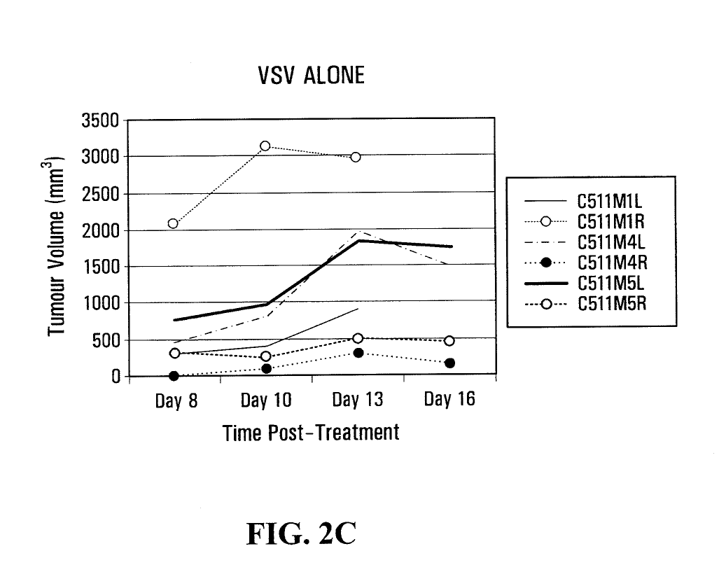

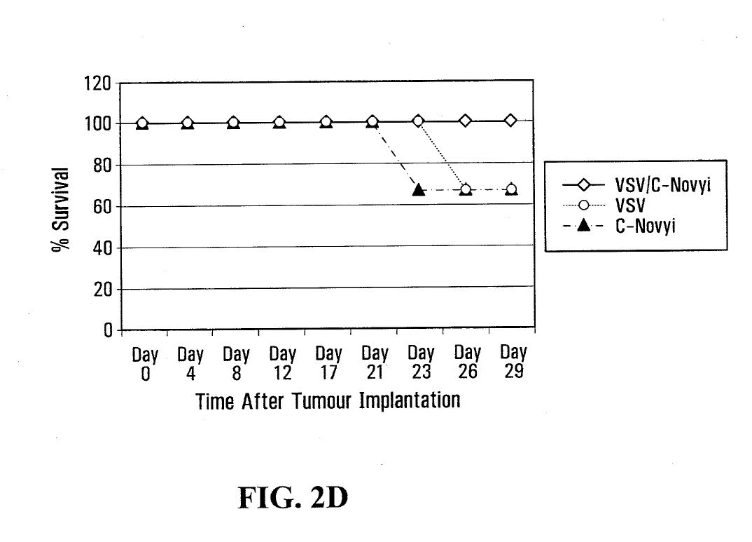

FIG. 2 shows the response of CT26 tumour bearing mice treated with VSV or C. Novyi, alone or in combination. In this example, Balb/c mice with CT26 subcutaneous tumours were treated intravenously with one dose of 10⁷ pfu D51-VSV plus 10⁴ C. Novyi spores (panel A), or with 10⁴ C. Novyi spores alone (panel B), or with 10⁷ pfu D51-VSV alone (panel C). Survival rates of treated mice are shown in panel D. The combination of VSV and C. Novyi demonstrated superior tumour responses than either agent alone.

Hematoxylin and eosin staining of CT26 tumours from Balb/c mice treated once with VSV or C. Novyi, alone or in combination illustrate a similar result. Balb/c mice with subcutaneous CT26 tumours were treated intravenously with D51-VSV or C. Novyi, alone or in combination. After 48 hours, tumours were harvested, frozen and stained with H&E. A tumour treated with 10⁸ C. Novyi spores showed extensive necrosis and substantial residual intact cells. A tumour treated with 5×10⁸ pfu of VSV showed no overt necrosis, and significant intact cellularity. Administration of C. Novyi before or after VSV leads to complete necrosis of solid tumours within 48 hours.

In combination, doses of VSV and C. Novyi can be dramatically reduced, and still lead to complete tumour necrosis. To illustrate this, Balb/c mice with subcutaneous CT26 tumours were treated intravenously with D51-VSV or C. Novyi, alone or in combination. After 48 hours, tumours were harvested, frozen and stained with H&E. A tumour treated with VSV alone shows no sign of necrosis. However, when VSV is administered concurrently with 10⁷ C. Novyi spores, significant necrosis is observed. The effect of the two agents in combination is so dramatic that reducing the doses of both agents by 1-2 log elicits extensive necrosis throughout the entire tumour.

### Example 5

### Anti-Clotting Treatments Prevent Tumor Vascular Shutdown Triggered by Oncolytic Virus Infection

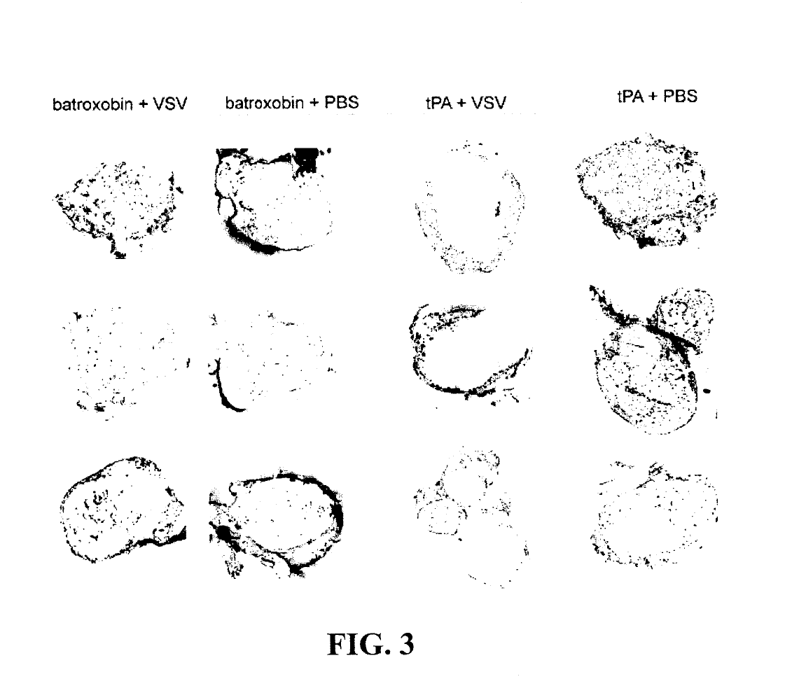

This Example relates to the observation that the lack of blood flow to a tumor in the inflammatory environment formed in the tumor as a consequence of oncolytic virus infection, as an effect or symptom of a host neutrophil response, is associated with blood clot formation (putatively triggered by inflammation). A component of the host neutrophil response appears, in at least some instances, to be accumulation of neutrophils in the tumor, causing localized activation of fibrinogen and formation of miniclots. In keeping with this, FIG. 3 illustrates embodiments in which anti-clotting treatments inhibit this aspect of the host neutrophil response, and prevent tumor vascular shutdown and promote virus spread. The illustrated results were obtained as follows. 6-8 week old Balb/C mice were injected subcutaneously with 3×10⁵ CT26 cells. Subcutaneous tumours were allowed to develop for 14 days. Mice were dosed intravenously with PBS or 5×10⁸ pfu D51 VSV GFP. Mice were treated intravenously with 4 mg/kg tPA or intraperitoneally with 20 U/kg batroxobin at the time of virus dosing and at 15 hours and 19 hours post virus treatment. At 24 h post virus infusion, mice were injected with 0.1 uM diameter fluorescent microspheres intravenously and sacrificed 5 min later. Tumours were excised, embedded in OCT medium and stored at −80C. Fluorescent microspheres were visualized on 10 uM sections with a ScanArray Express microarray scanner. Virus distribution was visualized by immunohistochemistry detecting VSV antigens using anti-VSV rabbit polyclonal serum.

The present results illustrate alternative embodiments with distinct modality and/or timing of the administration of anti-clotting agents to inhibit vascular shutdown. In one aspect of this Example, blood clots are disrupted by the thrombolytic agent tissue plasminogen activator (tPA), an agent that cleaves plasminogen to form plasmin. In another aspect, blood clot formation is prevented by treatment with batroxobin, a defibrinogenating agent. In the present Example, in the presence of tPA, vascular shutdown was observed in most VSV treated tumors, illustrating that in some embodiments thrombolytic agents such as tPA may be administered prior to the formation of blood clots in tumor vessels, for example so as to enhance perfusion of the tumor. In contrast, in mice treated with batroxobin, vascular shutdown was not observed in most VSV treated tumors, indicating that defibrinogenating agents such as batroxobin may be used in some embodiments concomitantly with viral dosing to ameliorate clot formation during VSV therapy, and to enhance tumor perfusion.

The present data augments the illustration, by immunohistochemical staining for VSV, that there is enhanced viral growth in tumors that do not exhibit vascular shutdown. Combining these aspects of the invention, in some embodiments, virus induced tumour vascular shutdown may be inhibited with a variety of anti-clotting agents, such as agents that inhibit the formation or persistence of clots in the tumor micro-vasculature, such as defibrinogenating enzymes. These aspects of the invention may be employed so as to increase virus spread within tumours. In an alternative aspect of the invention, the anti-clotting treatments may be discontinued, to initiate tumoricidal vascular shutdown.

In alternative embodiments, anti-clotting treatments may include treatments with thrombolytic or antithrombotic agents, such as tissue plasminogen activator (tPA), urokinase, prourokinase or streptokinase, heparinoids (such as danaparoid), defibrinating agents (such as Ancrod), direct thrombin inhibitors, hirudin and modifications or derivatives of these molecules. Alternatively, anti-clotting agents may be anticoagulants, such as heparin, low molecular weight heparins, warfarin, dicoumarol, aspirin, anisindone, phenindone, phenprocoumon, acenocoumarol, ethyl biscoumacetate and anisindionebishydroxy coumarin. Alternatively, anti-clotting agents may be anti-platelet agents, such as aspirin, dipyramidole, ticlopidine, and plavix.

### Example 6

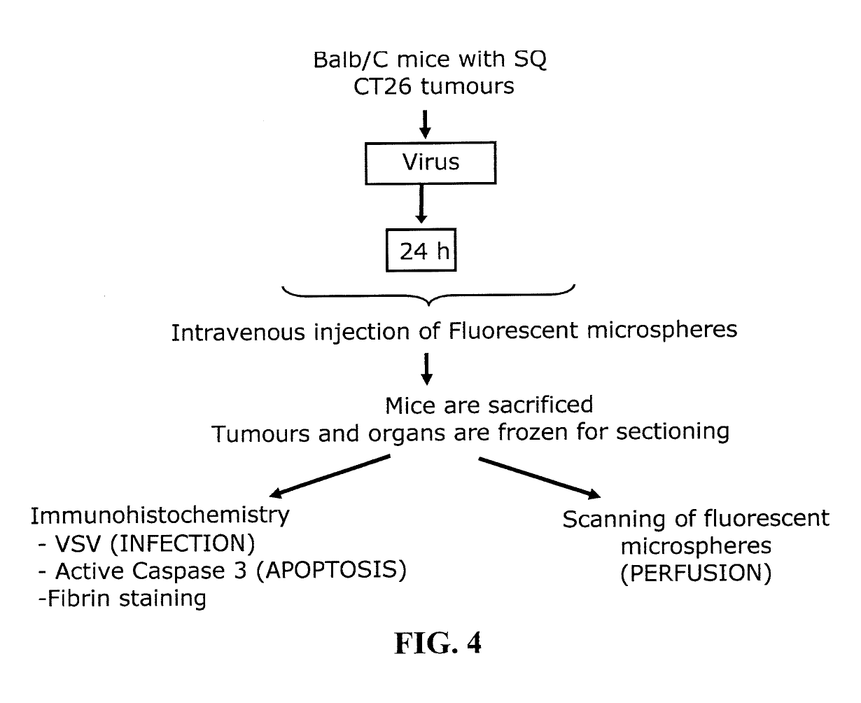

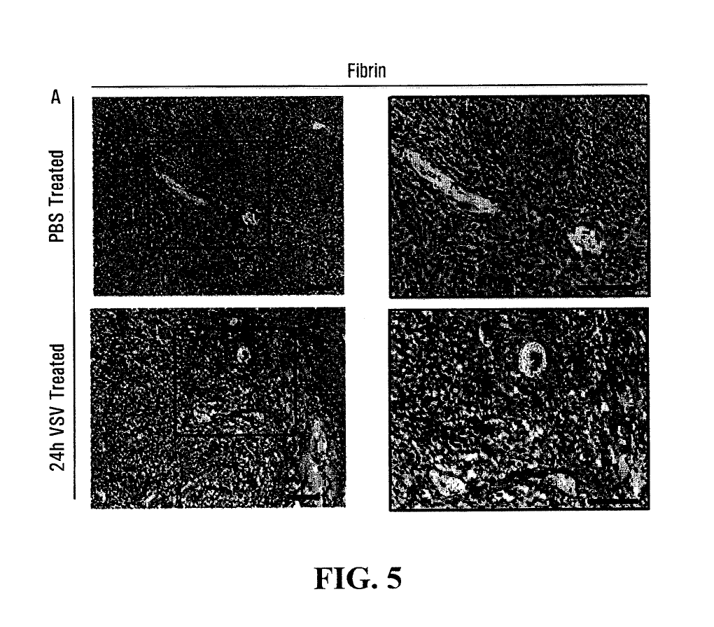

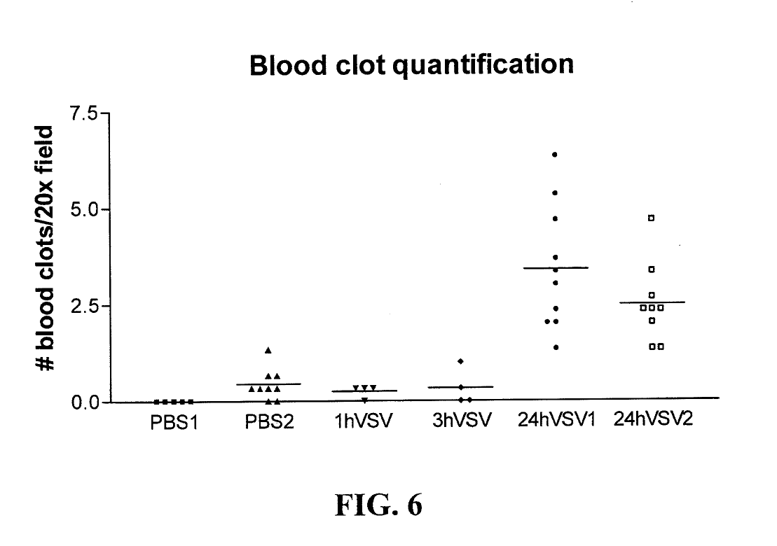

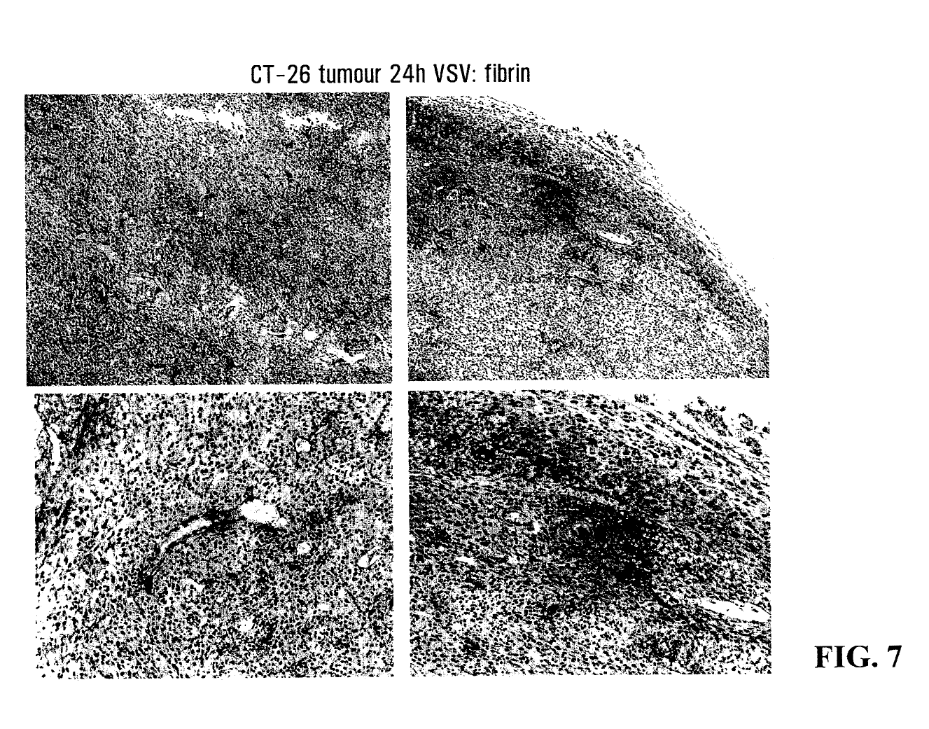

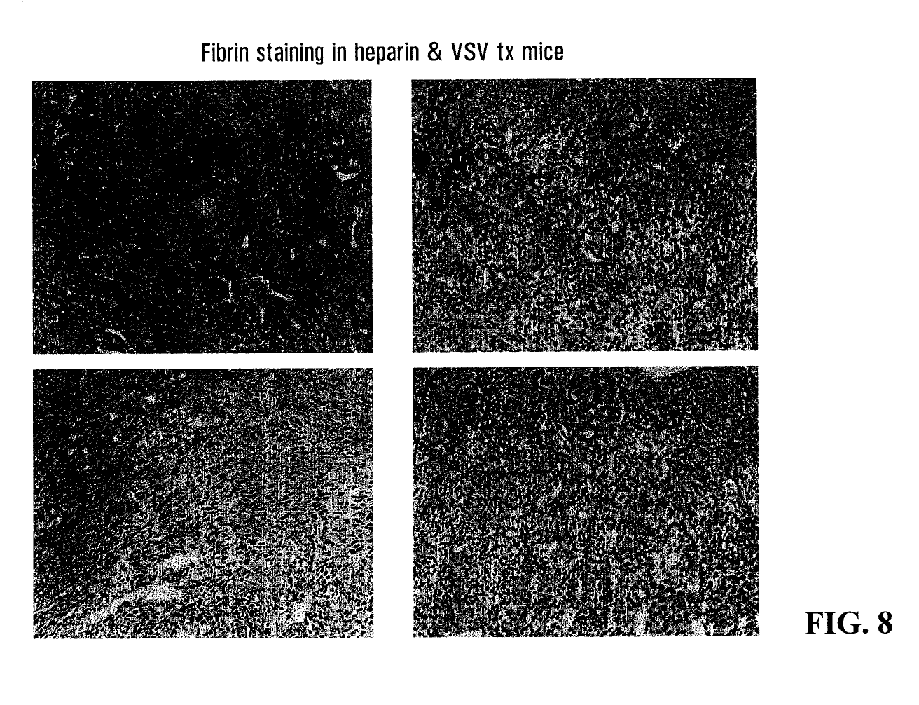

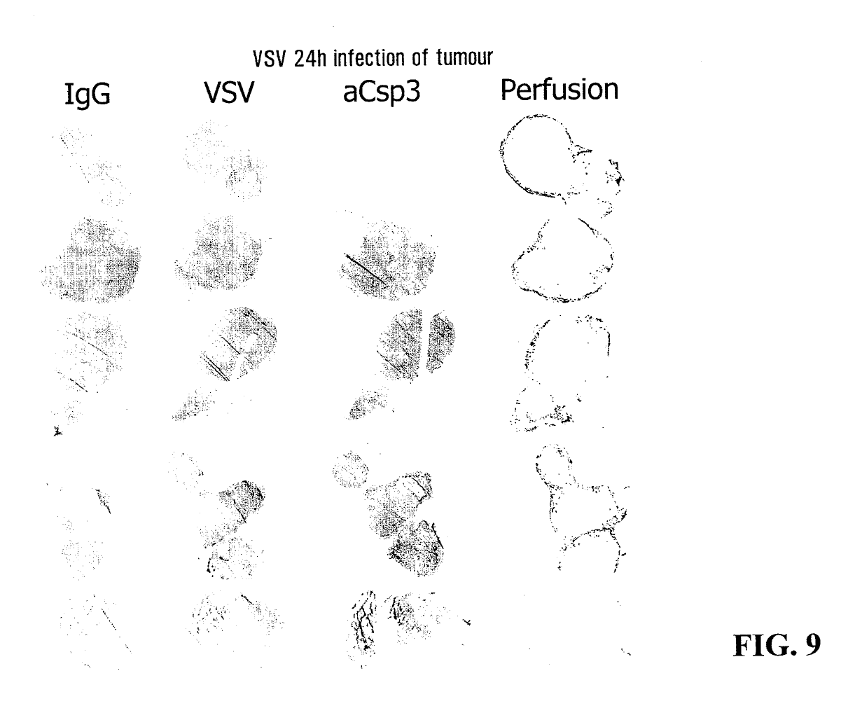

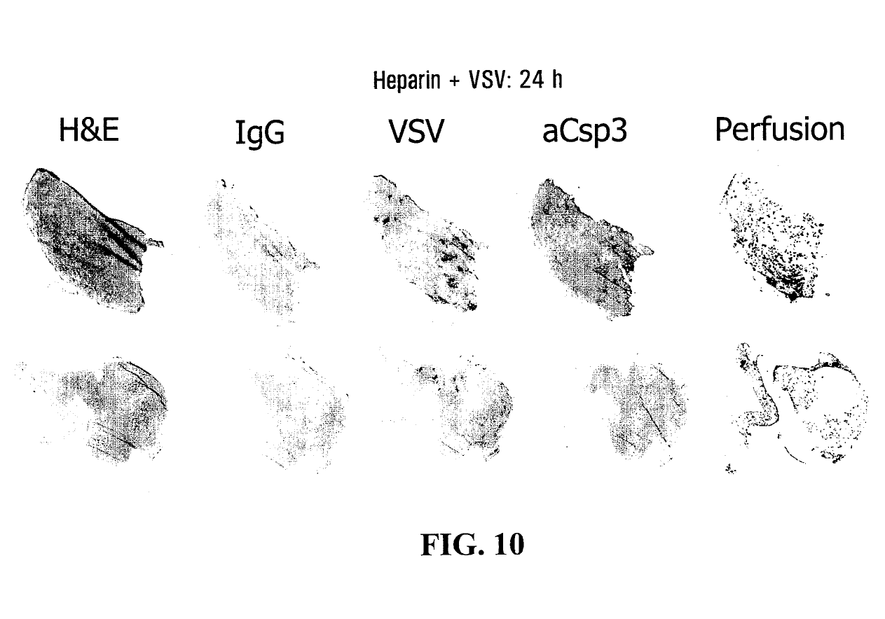

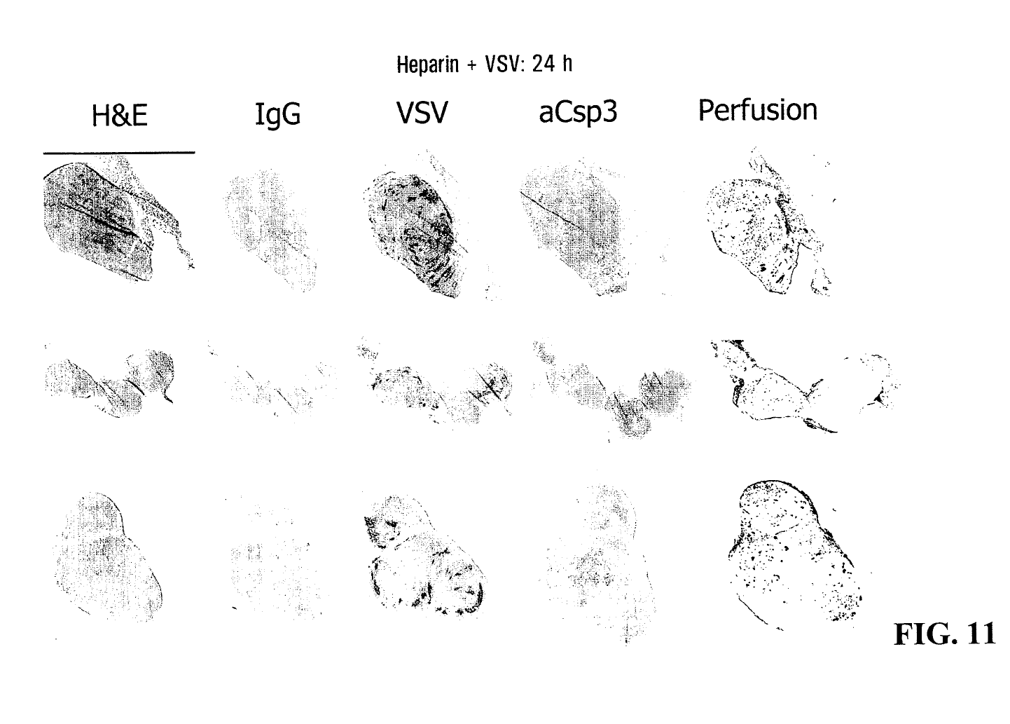

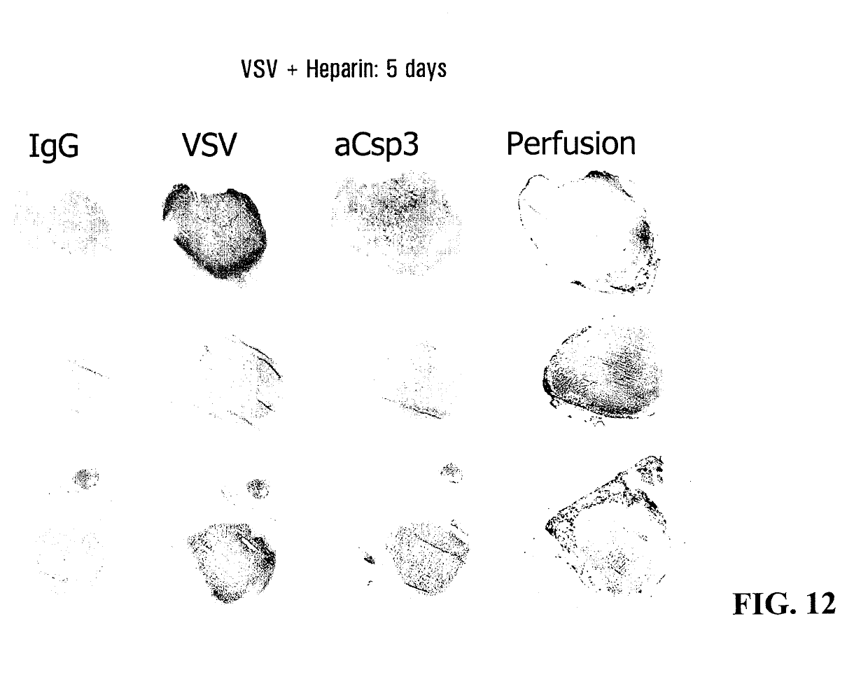

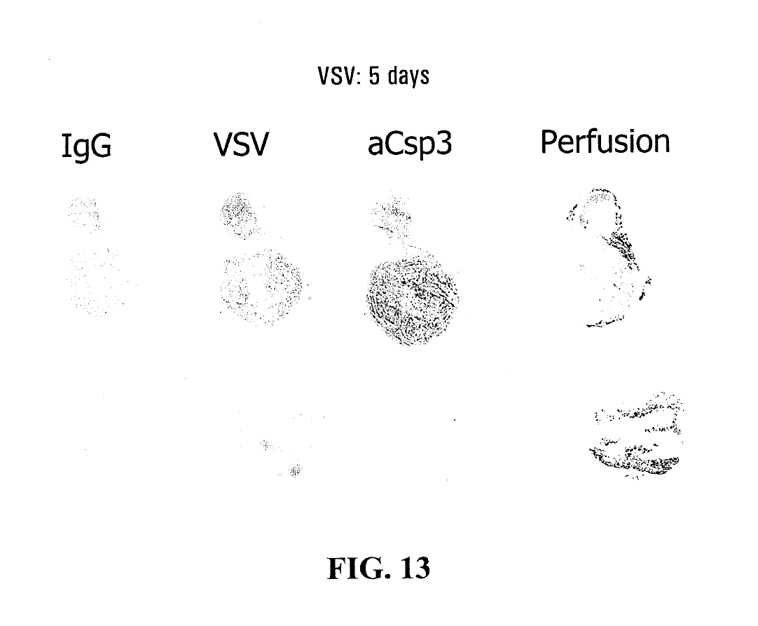

FIG. 4 is a schematic showing a protocol that illustrates the use of heparin to modulate tumour perfusion during oncolytic viral therapy. FIG. 5 shows comparative micrographs illustrating fibrin deposition in tumour vessels, demonstrating that when tumours are treated with an oncolytic virus (VSV), there is deposition of fibrin in the tumour and the tumour vasculature that leads to micro-vessel clot formation and loss of blood flow to the tumour. FIG. 6 is a graph illustrating the time frame of this oncolytic clot forming effect. FIGS. 7 and 8 are micrographs illustrating that if heparin is included with the virus in the same tumour treatment model, it blocks the deposition of fibrin and therefore clot formation. Comparison of the micrographs of tumour sections in FIGS. 9, 10 and 11 illustrates that blood flow (perfusion) is maintained in the tumor, and virus spread (labeled VSV) throughout the tumor is improved, if heparin is administered in conjunction with the virus. As shown in FIGS. 12 and 13, by day 5 there is evidence of significantly more virus replication in the tumour if heparin is included (FIG. 12), compared to tumours treated only with virus (FIG. 13). FIG. 14 illustrates treatment with heparin alone.

This Example illustrates the deferral of clot formation to facilitate viral spread, while also illustrating that the tumoricidal effects of vascular shut down are evident by day 5 even if heparin is included in the treatment, as illustrated by the absence of tumour perfusion by that time (which may be evidence of widespread tumour cell mortality on that time frame).

### REFERENCES

Bell et al. (2003) Getting oncolytic virus therapies off the ground. Cancer. Cell. July; 4(1):7-11.

Benjamim et al. (2005). Opposing and hierarchical roles of leukotrienes in local innate immune versus vascular responses in a model of sepsis. J. Immunol. 174(3): 1616-20.

Cairns et al. (2006) Overcoming physiologic barriers to cancer treatment by molecularly targeting the tumor microenvironment. Mol Cancer Res. 4(2):61-70.

Chiocca, E. A. et al. (2004). A phase I open-label, dose-escalation, multi-institutional trial of injection with an E1B-attenuated adenovirus, ONYX-015, into the peritumoral region of recurrent malignant gliomas, in the adjuvant setting. Mol. Ther. 10,958-966.

Croyle et al. (2001) Stealth” adenoviruses blunt cell-mediated and humoral immune responses against the virus and allow for significant gene expression upon readministration in the lung. J. Virol. 75,4792-4801.

Eltzschig H K, Collard C D. (2004) Vascular ischaemia and reperfusion injury. Br. Med. Bull. October 19; 70:71-86.

Endo, T. et al. (2002) In situ cancer vaccination with a replication-conditional HSV for the treatment of liver metastasis of colon cancer. Cancer Gene Ther. 9,142-148.

Eto, Y. et al. (2005) PEGylated adenovirus vectors containing RGD peptides on the tip of PEG show high transduction efficiency and antibody evasion ability. J. Gene Med. 7, 604-612.

Everts and van der Poel H G (2005). Replication-selective oncolytic viruses in the treatment of cancer. Cancer Gene Ther. February; 12(2):141-161.

Fisher, et al. (2001) Polymer-coated adenovirus permits efficient retargeting and evades neutralising antibodies. Gene Ther. 8, 341-348; 122.

Fukuhara, H. et al. (2003) Improvement of transduction efficiency of recombinant adenovirus vector conjugated with cationic liposome for human oral squamous cell carcinoma cell lines. Oral Oncol. 39, 601-609.

Grigoryants et al. (2005). Tamoxifen up-regulates catalase production, inhibits vessel wall neutrophil infiltration, and attenuates development of experimental abdominal aortic aneurysms. J. Vasc. Surg. 41(1): 108-14.

Grote et al. (2003). Neutrophils contribute to the measles virus-induced antitumor effect: enhancement by granulocyte macrophage colony-stimulating factor expression. Cancer Res. 63(19): 6463-8.

Harrow, S. et al. (2004). HSV1716 injection into the brain adjacent to tumour following surgical resection of high-grade glioma: safety data and long-term survival. Gene Ther. 11, 1648-1658.

Hirasawa, K. et al. (2003). Systemic reovirus therapy of metastatic cancer in immune-competent mice. Cancer Res. 63, 348-353.

Holterman, L. et al. (2004) Novel replication-incompetent vector derived from adenovirus type 11 (Ad11) for vaccination and gene therapy: low seroprevalence and non-crossreactivity with Ad5. J. Virol. 78, 13207-13215.

Huang et al., (2002). Fully Humanized Neutralizing Antibodies to Interleukin-8 (ABX-IL8) Inhibit Angiogenesis, Tumor Growth, and Metastasis of Human Melanoma. American Journal of Pathology. 2002; 161:125-134.

Hummel et al. (2005) The role of ICP0-null HSV-1 and interferon signaling defects in the effective treatment of breast adenocarcinoma. Mol. Ther. 31 Aug. 2005 (doi:10.1016/j.ymthe.2005.07.533).

Ichihashi, Y. (1996) Extracellular enveloped vaccinia virus escapes neutralization. Virology 217, 478-485.

Ikeda et al. (2002). The roles of IFN γ in protection against tumor development and cancer immunoediting. Cytokine Growth Factor Rev. 13, 95-109.

Ikeda, K. et al. (1999) Oncolytic virus therapy of multiple tumors in the brain requires suppression of innate and elicited antiviral responses. Nature Med. 5, 881-887.

Ilan, Y. et al. (1997) Transient immunosuppression with FK506 permits long-term expression of therapeutic genes introduced into the liver using recombinant adenoviruses in the rat. Hepatology 26, 949-956.

Jablonska et al. (2002). Priming effects of GM-CSF, IFN-gamma and TNF-alpha on human neutrophil inflammatory cytokine production. Melanoma Res. 12(2): 123-8.

Jiang D, et al. (2005) Regulation of lung injury and repair by Toll-like receptors and hyaluronan. Nat. Med. November; 11(11):1173-1179.

Jooss et al. (1996) Cyclophosphamide diminishes inflammation and prolongs transgene exand decoy receptors for CXCR2 ligandspression following delivery of adenoviral vectors to mouse liver and lung. Hum. Gene Ther. 7, 1555-1566.

Kaufman, H. L. et al. (2005). Targeting the local tumor microenvironment with vaccinia virus expressing B7.1 for the treatment of melanoma. J. Clin. Invest. 115, 1903-1912.

Kokura S, et al. (2002) Anoxia/reoxygenation-induced leukocyte-endothelial cell interactions. Free Radic. Biol. Med. August 15; 33(4):427-432.

Kuriyama, S. et al. (1999) Transient cyclophosphamide treatment before intraportal readministration of an adenoviral vector can induce re-expression of the original gene construct in rat liver. Gene Ther. 6, 749-757.

Law, M. & Smith, G. L. (2001) Antibody neutralization of the extracellular enveloped form of vaccinia virus. Virology 280, 132-142.

Li and Gordon (2001) Il-8((3-73))K11R is a high affinity agonist of the neutrophil CXCR1 and CXCR2. Biochem Biophys Res Commun. 2001 Aug. 24; 286(3):595-600.

Li et al. (2002). CXCL8((3-73))K11R/G31P antagonizes the neutrophil chemoattractants present in pasteurellosis and mastitis lesions and abrogates neutrophil influx into intradermal endotoxin challenge sites in vivo. Vet Immunol lmmunopathol. 2002 November; 90(1-2):65-77.

Londhe et al. (2005). CXCR2/CXCR2 ligand biological axis impairs alveologenesis during dsRNA-induced lung inflammation in mice. Pediatr. Res. 58(5): 919-26.

Lorence, R. M. et al. (2003). Overview of phase I studies of intravenous administration of PV701, an oncolytic virus. Curr. Opin. Mol. Ther. 5, 618-624.

Lorence, R. M. et al. (2003). Overview of phase I studies of intravenous administration of PV701, an oncolytic virus. Curr. Opin. Mol. Ther. 5, 618-624;

Lorence, R. M. et al. (2005). Continuing the interaction between non-clinical and clinical studies. Third International Meeting on Oncolytic Virus Therapeutics: Banff, Alberta (12 Mar. 2005);

Lorence, R. M. et al. (2005). Continuing the interaction between non-clinical and clinical studies. Third International Meeting on Oncolytic Virus Therapeutics: Banff, Alberta (12 Mar. 2005).

Lunt et al (2005). Tirapazamine Administered as a Neoadjuvant to Radiotherapy Reduces Metastatic Dissemination. Clinical Cancer Research Vol. 11, 4212-4216.

Mastrangelo, M. J. et al. (1999) Intratumoral recombinant GM-CSF-encoding virus as gene therapy in patients with cutaneous melanoma. Cancer Gene Ther. 6(5), 409-422.

McCart et al. (2001). Systemic Cancer Therapy with a Tumor-selective Vaccinia Virus Mutant Lacking Thymidine Kinase and Vaccinia Growth Factor Genes. Cancer Research 61, 8751-8757)

McCart et al. (2001). Systemic Cancer Therapy with a Tumor-selective Vaccinia Virus Mutant Lacking Thymidine Kinase and Vaccinia Growth Factor Genes. Cancer Research 61, 8751-8757.

McClenahan et al. (2000). Role of inflammatory mediators in priming, activation, and deformability of bovine neutrophils. Am. J. Vet. Res. 61(5): 492-8.

Mian et al. (2003) Fully Human Anti-Interleukin 8 Antibody Inhibits Tumor Growth in Orthotopic Bladder Cancer Xenografts via Down-Regulation of Matrix Metalloproteases and Nuclear Factor-{kappa}B Clin. Cancer Res., Aug. 1, 2003; 9(8): 3167 - 3175.

Morgan et al. (2005) Can neutrophils be manipulated in vivo? Rheumatology 2005 44(5):597-601.

Myers, R. et al. (2005). Oncolytic activities of approved mumps and measles vaccines for therapy of ovarian cancer. Cancer Gene Ther. 12, 593-599.

Okunieff et al. (2005) Past, present, and future of oxygen in cancer research. Adv Exp Med Biol. 566:213-22.

Onai et al. (2003). Blockade of cell adhesion by a small molecule selectin antagonist attenuates myocardial ischemia/reperfusion injury. Eur. J. Pharmacol. 481(2-3): 217-25.

Parato et al. (2005). Recent progress in the battle between oncolytic viruses and tumours. Nat Rev Cancer. 5(12): 965-76.

Pecora, A. L. et al. (2002) Phase I trial of intravenous administration of PV701, an oncolytic virus, in patients with advanced solid cancers. J. Clin. Oncol. 20, 2251-2266.

Pecora, A. L. et al. (2002) Phase I trial of intravenous administration of PV701, an oncolytic virus, in patients with advanced solid cancers. J. Clin. Oncol. 20, 2251-2266.

Pecora, A. L. et al. (2002) Phase I trial of intravenous administration of PV701, an oncolytic virus, in patients with advanced solid cancers. J. Clin. Oncol. 20, 2251-2266;

Reid et al. (2002). Intravascular adenoviral agents in cancer patients: lessons from clinical trials. Cancer Gene Ther. 9, 979-986.

Reid et al. (2002). Intravascular adenoviral agents in cancer patients: lessons from clinical trials. Cancer Gene Ther. 9, 979-986.

Reid, T. et al. (2001). Intra-arterial administration of a replication selective adenovirus (dl1520) in patients with colorectal carcinoma metastatic to the liver: a phase I trial. Gene Ther. 8, 1618-1626;

Reid, T. et al. (2001). Intra-arterial administration of a replication selective adenovirus (dl1520) in patients with colorectal carcinoma metastatic to the liver: a phase I trial. Gene Ther. 8, 1618-1626;

Ries S J, Brandts C H. (2004) Oncolytic viruses for the treatment of cancer: current strategies and clinical trials. Drug Discov. Today 2004 Sep. 1; 9(17):759-768

Shah et al., (2003). Oncolytic viruses: clinical applications as vectors for the treatment of malignant gliomas. J. Neurooncol. 65, 203-226.

Shah, et al. (2003) Oncolytic viruses: clinical applications as vectors for the treatment of malignant gliomas. J. Neurooncol. 65, 203-226

Smith et al. (1996). Transient immunosuppression permits successful repetitive intravenous administration of an adenovirus vector. Gene Ther. 3, 496-502.

Souza et al. (2003). Role of PAF receptors during intestinal ischemia and reperfusion injury. A comparative study between PAF receptor-deficient mice and PAF receptor antagonist treatment. Br. J. Pharmacol. 139(4) 733-40.

Souza et al. (2004) Repertaxin, a novel inhibitor of rat CXCR2 function, inhibits inflammatory responses that follow intestinal ischaemia and reperfusion injury. Br J Pharmacol. (2004) 143(1):132-42.

Stojdl, D. F. et al. (2000). Exploiting tumor-specific defects in the interferon pathway with a previously unknown oncolytic virus. Nature Med. 6, 821-825.

Stojdl, D. F. et al. (2003) VSV strains with defects in their ability to shutdown innate immunity are potent systemic anti-cancer agents. Cancer Cell 4, 263-275.

Takai et al. (2005) Blockade of Neutrophil Elastase Attenuates Severe Liver Injury in Hepatitis B Transgenic Mice. J Virol. (2005) 79(24):15142-50.

Tazawa et al. (2003). Infiltration of neutrophils is required by acquisition of metastatic phenotype of benign murine fibrosarcoma cells: implication of inflammation-associated carcinogenesis and tumor progression. Am. J. Pathol, 163(6): 2221-32.

Toda et al. (1999) Herpes simplex virus as an in situ cancer vaccine for the induction of specific anti-tumor immunity. Hum. Gene Ther. 10, 385-393.

Toda et al. (2002) Immuno-viral therapy of brain tumors by combination of viral therapy with cancer vaccination using a replication-conditional HSV. Cancer Gene Ther. 9, 356-364

Vinten-Johansen J. (2004) Involvement of neutrophils in the pathogenesis of lethal myocardial reperfusion injury. Cardiovasc.Res. 2004 Feb. 15; 61(3):481-497

Wakimoto et al. (2004) Altered expression of antiviral cytokine mRNAs associated with cyclophosphamide's enhancement of viral oncolysis. Gene Ther. 11, 214-23.

Widdowson et al. (2004) Evaluation of Potent and Selective Small-Molecule Antagonists for the CXCR2 Chemokine Receptor J Med Chem 47:1319-21.

Williams et al. (2005) Exogenous and endogenous markers of tumour oxygenation status: definitive markers of tumour hypoxia? Adv Exp Med Biol. 566:285-94.

Xia, Z. J. et al. (2004). Phase Ill randomized clinical trial of intratumoral injection of E1B gene-deleted adenovirus (H101) combined with cisplatin-based chemotherapy in treating squamous cell cancer of head and neck or esophagus. Ai Zheng 23, 1666-1670.

Yasui et al. (2005). Thalidomide as an immunotherapeutic agent: the effects on neutophil-mediated inflammation. Curr. Pharm. Des. 11(3): 395-401.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as described herein, with reference to the examples and drawings.

1. A method of treating a solid tumor in a host, comprising:
infecting the tumor with an amount of one or more strains of oncolytic virus effective to cause a lytic infection of tumor cells within the tumor;
modulating a host neutrophil response to the lytic infection, so that during the course of the lytic infection, the host has an initial neutrophil response and a secondary neutrophil response, wherein the secondary neutrophil response mediates a greater degree of apoptotic killing of tumor cells than does the initial neutrophil response.

2. The method of claim 1, further comprising suppressing the host neutrophil response to the lytic infection, so that the host has a suppressed neutrophil condition during the initial neutrophil response, so as to increase the number of tumor cells infected with the virus during the initial neutrophil response compared to the number that would be infected in the absence of the step of suppressing the host neutrophil response.

3. The method of claim 2, wherein suppressing the host neutrophil response to the lytic infection is carried out so as to inhibit neutrophil mediated vascular shutdown in the tumor.

4. The method of claim 2, further comprising releasing the suppression of the host neutrophil response during the course of the lytic infection to initiate the secondary neutrophil response, so as to facilitate neutrophil mediated inflammation in the tumor during the secondary neutrophil response that results in apoptotic killing of tumor cells.

5. The method of claim 2, further comprising stimulating the host neutrophil response during the course of the lytic infection to augment the secondary neutrophil response, so as to enhance neutrophil mediated inflammation in the tumor that results in apoptotic killing of tumor cells.

6. The method of claim 1, wherein the oncolytic virus mediates expression of a neutrophil modulating protein that modulates the host neutrophil response.

7. The method of claim 2, wherein the oncolytic virus mediates expression of a neutrophil suppressing protein that suppresses the host neutrophil response.

8. The method of claim 5, wherein the oncolytic virus mediates expression of a neutrophil stimulating protein that stimulates the host neutrophil response.

9. (canceled)

10. The method of claim 2, wherein an effective amount of a neutrophil suppressing agent is administered to the host to suppress the host neutrophil response.

11. The method of claim 5, wherein an effective amount of a neutrophil stimulating agent is administered to the host to stimulate the host neutrophil response.

12. The method of claim 10, wherein the neutrophil suppressing agent is selected from the group consisting of: neutrophil inhibitory factor (NAF); glucosamine salts; agonists of MMP-9; peptide antagonists of NAF; antibodies that bind to the CD11 b subunit of neutrophil integrin Mol; an anti-granulocyte antibody; a selectin antagonist; inhibitors of chemokine ligand/receptor complex CXCR2UCXCR2; thalidomide; tamoxifen; a cysteinyl-leukotriene receptor antagonist; a platelet activating factor-receptor antagonist; IL-6; IL-6 and TGFP;
diethylmaleate (DEM); phorone; buthionine-sulfoximine (BSO); glutathione depleting diethylmaleate (DEM) mimetics; glutathione depleting phorone mimetics; glutathione depleting buthionine sulfoximine (BSO) mimetics; peptides having the formula f-Met-Leu-X, where X is selected from the group consisting of Tyr, Tyr-Phe, PhePhe and Phe-Tyr; proteins having the I-Domain from the human leukocyte beta2-integrin Mac-1; Duffy antigen receptor for chemokines (DARC); and decoy receptors for CXCR2 ligands.

13. The method of claim 11, wherein the neutrophil stimulating agent is selected from the group consisting of: neutrophil-activating immune response modifier (IRM); a toll-like receptor (TLR)-8-selective agonist; neutrophil-activating factor; ENA-78; medium-chain fatty acids; glycerides; protein S100A8; protein S100A9; protein S100A12; protein S100A8/A9; GM-CSF; interferon-y; tumor necrosis factor (TNF)-a; PAF; IL-8; Gro-alpha.

14. The method of claim 1, wherein the oncolytic virus is selected from the group consisting of adenovirus; reovirus; herpes simplex virus, such as HSV1; Newcastle disease virus; vaccinia virus; Coxsackievirus; measles virus; vesicular stomatitis virus (VSV); influenza virus; myxoma virus; Rhabdovirus, picornavirus.

15. The method of claim 1, further comprising administering to the host a chemotherapeutic agent to augment killing of tumor cells during the secondary neutrophil response.

16. The method of claim 15, wherein the chemotherapeutic agent is an agent that preferentially kills hypoxic tumor tissues.

17. (canceled)

18. The method of claim 1, wherein the solid tumor is of a cancer selected from the group consisting of: carcinomas of breast, colon, rectum, lung, oropharynx, hypopharynx, esophagus, stomach, pancreas, liver, gallbladder, bile ducts, small intestine, urinary tract, kidney, bladder, urothelium, female genital tract, cervix, uterus, ovaries, male genital tract, prostate, seminal vesicles, testes, endocrine glands, thyroid, adrenal, pituitary gland, and skin; germ cell tumors; choriocarcinoma; gestational trophoblastic disease; hemangiomas; melanomas; sarcomas; tumors of the brain, nerves, eyes, and meninges; astrocytomas; gliomas; glioblastomas; retinoblastomas; neuromas; neuroblastomas; Schwannomas; meningiomas; and solid tumors arising from hematopoietic malignancies.

19. The method of claim 1, wherein the oncolytic virus is administered to the host systemically to infect the tumor.

20. (canceled)

21. (canceled)

22. The method of claim 1, wherein the oncolytic virus and the neutrophil modulating agent are co-administered to the host.

23. (canceled)

24. (canceled)

25. (canceled)

26. (canceled)

27. A method of treating a solid tumor in a host, comprising:
infecting the tumor with an amount of one or more strains of oncolytic virus effective to cause a lytic infection of tumor cells within the tumor;
administering an effective amount of an anti-clotting agent to the host, so as to inhibit neutrophil mediated vascular shutdown in the tumor.

28. The method of claim 27, wherein the anti-clotting agent is a thrombolytic agent, an antithrombotic agent, a fibrinolytic agent, an anticoagulant or an anti-platelet agent.

29. (canceled)

30. The method of claim 27, further comprising discontinuing the administration of the anti-clotting agent so as to augment neutrophil mediated vascular shutdown in the tumor.

31. The method of claim 27, further comprising modulating a host neutrophil response to the lytic infection, so that during the course of the lytic infection, the host has an initial neutrophil response and a secondary neutrophil response, wherein the secondary neutrophil response mediates a greater degree of apoptotic killing of tumor cells than does the initial neutrophil response.

32. The method of claim 31, further comprising suppressing the host neutrophil response to the lytic infection, so that the host has a suppressed neutrophil condition during the initial neutrophil response, so as to increase the number of tumor cells infected with the virus during the initial neutrophil response compared to the number that would be infected in the absence of the step of suppressing the host neutrophil response.

33. The method of claim 32, wherein suppressing the host neutrophil response to the lytic infection is carried out so as to inhibit neutrophil mediated vascular shutdown in the tumor.

34. The method of claim 32, further comprising releasing the suppression of the host neutrophil response during the course of the lytic infection to initiate the secondary neutrophil response, so as to facilitate neutrophil mediated inflammation in the tumor during the secondary neutrophil response that results in apoptotic killing of tumor cells.

35. The method of claim 31, further comprising stimulating the host neutrophil response during the course of the lytic infection to augment the secondary neutrophil response, so as to enhance neutrophil mediated inflammation in the tumor that results in apoptotic killing of tumor cells.

36. The method of claim 31, wherein the oncolytic virus mediates expression of a neutrophil modulating protein that modulates the host neutrophil response.

37. The method of claim 32, wherein the oncolytic virus mediates expression of a neutrophil suppressing protein that suppresses the host neutrophil response and/or a neutrophil stimulating protein that stimulates the host neutrophil response.

38. (canceled)

39. (canceled)

40. The method of claim 32, wherein an effective amount of a neutrophil suppressing agent is administered to the host to suppress the host neutrophil response.

41. The method of claim 35, wherein an effective amount of a neutrophil stimulating agent is administered to the host to stimulate the host neutrophil response.

42. The method of claim 40, wherein the neutrophil suppressing agent is selected from the group consisting of: neutrophil inhibitory factor (NAF); glucosamine salts; agonists of MMP-9; peptide antagonists of NAF; antibodies that bind to the CD11 b subunit of neutrophil integrin Mol; an anti-granulocyte antibody; a selectin antagonist; inhibitors of chemokine ligand/receptor complex CXCR2UCXCR2; thalidomide; tamoxifen; a cysteinyl-leukotriene receptor antagonist; a platelet activating factor-receptor antagonist; IL-6; IL-6 and TGF13; diethylmaleate (DEM); phorone; buthionine-sulfoximine (BSO); glutathione depleting diethylmaleate (DEM) mimetics; glutathione depleting phorone mimetics; glutathione depleting buthionine sulfoximine (BSO) mimetics; peptides having the formula f-Met-Leu-X, where X is selected from the group consisting of Tyr, Tyr-Phe, PhePhe and Phe-Tyr; proteins having the I-Domain from the human leukocyte beta2-integrin Mac-1; Duffy antigen receptor for chemokines (DARC); decoy receptors for CXCR2 ligands; antineutrophil antibodies; CAMPATH (anti CD52); an anti-integrin antibody; a myelosuppressive chemotherapeutic agent; cyclophosphamide; anthracycline; an anti-inflammatory; a COX inhibitor; ASA; ibuprofen; and naproxyn.

43. The method of claim 41, wherein the neutrophil stimulating agent is selected from the group consisting of: neutrophil-activating immune response modifier (IRM); a toll-like receptor (TLR)-8-selective agonist; neutrophil-activating factor; ENA-78; medium-chain fatty acids; glycerides; protein S100A8; protein S100A9; protein S100A12; protein S100A8/A9; GM-CSF; interferon-y; tumor necrosis factor (TNF)-a; PAF; IL-8; Gro-alpha.

44. The method of claim 31, wherein the oncolytic virus is selected from the group consisting of adenovirus; reovirus; herpes simplex virus, such as HSV1; Newcastle disease virus; vaccinia virus; Coxsackievirus; measles virus; vesicular stomatitis virus (VSV); influenza virus; myxoma virus; Rhabdovirus, picornavirus.

45. The method of claim 31, further comprising administering to the host a chemotherapeutic agent to augment killing of cells during the secondary neutrophil response.

46. The method of claim 44, wherein the chemotherapeutic agent is an agent that preferentially kills hypoxic tumor tissues.

47. The method of claim 45 or 16, wherein the chemotherapeutic agent is selected from the group consisting of: dihydropyrimido-quinoxalines; dihydropyrimido-pyridopyrazines; quinoxaline derivatives; pyridopyrazine derivatives; 1,2-dihydro-8-piperazinyl-4-phenylimidazopyridopyrazine oxides; 1,2-dihydro-8-piperazinyl-4-phenylimidazo quinoxaline oxides; nitrophenyl mustard; nitrophenylaziridine alcohols; anthraquinone compounds; butylene amine oxime ring compounds and radiometal complexes thereof; 1,2,4-benzotriazine-1,4-dioxide compounds.

48. The method of claim 31, wherein the solid tumor is of a cancer selected from the group consisting of: carcinomas of breast, colon, rectum, lung, oropharynx, hypopharynx, esophagus, stomach, pancreas, liver, gallbladder, bile ducts, small intestine, urinary tract, kidney, bladder, urothelium, female genital tract, cervix, uterus, ovaries, male genital tract, prostate, seminal vesicles, testes, endocrine glands, thyroid, adrenal, pituitary gland, and skin; germ cell tumors; choriocarcinoma; gestational trophoblastic disease; hemangiomas; melanomas; sarcomas; tumors of the brain, nerves, eyes, and meninges; astrocytomas; gliomas; glioblastomas; retinoblastomas; neuromas; neuroblastomas; Schwannomas; meningiomas; and solid tumors arising from hematopoietic malignancies.

49. The method of claim 31, wherein the oncolytic virus is administered to the host systemically to infect the tumor.

50. (canceled)

51. (canceled)

52. The method of claim 27, wherein the oncolytic virus and the neutrophil modulating agent are co-administered to the host.

53. (canceled)

54. (canceled)

55. (canceled)

56. (canceled)


SEQUENCE LISTING (24 sequences)

SEQ ID NO: 1 - DNA, 20 bp, Artificial
  Left primer sequence for M-csf gene
ctggaaggaggatcagcaag

SEQ ID NO: 2 - DNA, 20 bp, Artificial
  Right primer sequence for M-csf gene
ccccacagaagaatccaatg

SEQ ID NO: 3 - DNA, 20 bp, Artificial
  Left primer sequence for Cox2 gene
tcctcctggaacatggactc

SEQ ID NO: 4 - DNA, 20 bp, Artificial
  Right primer sequence for Cox2 gene
ccccaaagatagcatctgga

SEQ ID NO: 5 - DNA, 20 bp, Artificial
  Left primer sequence for C3 gene
ctgtgtgggtggatgtgaag

SEQ ID NO: 6 - DNA, 20 bp, Artificial
  Right primer sequence for C3 gene
ttggtgcactcaagatctgc

SEQ ID NO: 7 - DNA, 20 bp, Artificial
  Left primer sequence for CXCL1 gene
gctgggattcacctcaagaa

SEQ ID NO: 8 - DNA, 19 bp, Artificial
  Right primer sequence for CXCL1 gene
gtcagaagccagcgttcac

SEQ ID NO: 9 - DNA, 20 bp, Artificial
  Left primer sequence for CXCL2 gene
tccagagcttgagtgtgacg

SEQ ID NO: 10 - DNA, 20 bp, Artificial
  Right primer sequence for CXCL2 gene
aggcacatcaggtacgatcc

SEQ ID NO: 11 - DNA, 20 bp, Artificial
  Left primer sequence for Il-6 gene
aacgatgatgcacttgcaga

SEQ ID NO: 12 - DNA, 20 bp, Artificial
  Right primer sequence for Il-6 gene
ggaaattggggtaggaagga

SEQ ID NO: 13 - DNA, 20 bp, Artificial
  Left primer sequence for IFN beta gene
ataagcagctccagctccaa

SEQ ID NO: 14 - DNA, 20 bp, Artificial
  Right primer sequence for IFN beta gene
ctgtctgctggtggagttca

SEQ ID NO: 15 - DNA, 20 bp, Artificial
  Left primer sequence for Vcam1 gene
gtggtgctgtcacaatgacc

SEQ ID NO: 16 - DNA, 20 bp, Artificial
  Right primer sequence for Vcam1 gene
acgtcagaacaaccgaatcc

SEQ ID NO: 17 - DNA, 20 bp, Artificial
  Left primer sequence for E-selectin gene
ccagtgcttctggacctttc

SEQ ID NO: 18 - DNA, 20 bp, Artificial
  Right primer sequence for E-selectin gene
caagctaaagccctcattgc

SEQ ID NO: 19 - DNA, 20 bp, Artificial
  Left primer sequence for Il-1 beta gene
caggcaggcagtatcactca

SEQ ID NO: 20 - DNA, 20 bp, Artificial
  Right primer sequence for Il-1 beta gene
agctcatatgggtccgacag

SEQ ID NO: 21 - DNA, 20 bp, Artificial
  Left primer sequence for TNF alpha gene
ccacatctccctccagaaaa

SEQ ID NO: 22 - DNA, 20 bp, Artificial
  Right primer sequence for TNF alpha gene
agggtctgggccatagaact

SEQ ID NO: 23 - DNA, 20 bp, Artificial
  Left primer sequence for CAT gene
gcgtgttacggtgaaaacct

SEQ ID NO: 24 - DNA, 20 bp, Artificial
  Right primer sequence for CAT gene
gggcgaagaagttgtccata